(19) United States
(12) Patent Application Publication
(10) Pub. No.: US 2015/0361464 A1
(43) Pub. Date: Dec. 17, 2015

(54) METHODS, REAGENTS AND CELLS FOR BIOSYNTHESIZING COMPOUNDS

(71) Applicant: INVISTA North America S.á r.l., Wilmington, DE (US)

(72) Inventors: Adriana Leonora Botes, Rosedale East (GB); Alex Van Eck Conradie, Eaglescliffe (GB)

(21) Appl. No.: 14/740,933

(22) Filed: Jun. 16, 2015

Related U.S. Application Data
(60) Provisional application No. 62/012,585, filed on Jun. 16, 2014.

(51) Int. Cl. C12P 13/00; C12P 7/44; C12P 7/42; C12N 9/10; C12N 9/88; C12N 9/06; C12N 9/80; C12N 9/02; C07C 229/08; C12P 7/18

(52) U.S. Cl. CPC C12P 13/001; C07C 229/08; C12P 7/44; C12P 7/42; C12P 7/18; C12N 9/88; C12N 9/0022; C12N 9/80; C12N 9/0069; C12N 9/1096; C12N 9/0008; C12Y 401/01; C12Y 104/03021; C12Y 305/0103; C12Y 113/12002; C12Y 206/01; C12Y 102/01003; C12Y 102/01004

(57) ABSTRACT

This document describes biochemical pathways for producing glutaric acid, 5-aminopentanoic acid, 5-hydroxypentanoic acid or 1,5-pentanediol by forming one or two terminal functional groups, comprised of carboxyl, amine or hydroxyl group, in a C5 backbone substrate such as cadaverine or 5-aminopentanamide.

### CROSS-REFERENCE TO RELATED APPLICATIONS

This application claims the benefit of U.S. Application No. 62/012,585, filed on Jun. 16, 2014, which is incorporated by reference herein in its entirety.

### TECHNICAL FIELD

This invention relates to methods for biosynthesizing glutaric acid, 5-aminopentanoic acid, cadaverine, 5-hydroxypentanoic acid, or 1,5-pentanediol (hereafter “C5 building blocks”) using one or more isolated enzymes such as reductases, monooxygenases, decarboxylases, amidases, oxidases, dehydrogenases, or ω-transaminases, and recombinant hosts that produce such C5 building blocks.

### BACKGROUND

Nylons are polyamides which are generally synthesized by the condensation polymerization of a diamine with a dicarboxylic acid. Similarly, Nylons may be produced by the condensation polymerization of lactams. A ubiquitous nylon is Nylon 6,6, which is produced by condensation polymerization of hexamethylenediamine (HMD) and adipic acid. Nylon 6 can be produced by a ring opening polymerization of caprolactam (Anton & Baird, Polyamides Fibers, Encyclopedia of Polymer Science and Technology, 2001).

Nylon 5, Nylon 5,5 and other variants including C5 monomers represent novel polyamides with value-added characteristics compared to Nylon 6 and Nylon 6,6 in a number of applications. Nylon 5 is produced by polymerisation of 5-aminopentanoic acid, whereas Nylon 5,5 is produced by condensation polymerisation of glutaric acid and cadaverine. No economically viable petrochemical routes exist to producing the monomers for Nylon 5 and Nylon 5,5.

Given no economically viable petrochemical monomer feedstocks, biotechnology offers an alternative approach via biocatalysis. Biocatalysis is the use of biological catalysts, such as enzymes, to perform biochemical transformations of organic compounds.

Both bioderived feedstocks and petrochemical feedstocks are viable starting materials for the biocatalysis processes.

Accordingly, against this background, it is clear that there is a need for sustainable methods for producing one or more of glutaric acid, 5-hydroxypentanoate, 5-aminopentanoate, cadaverine, and 1,5-pentanediol (hereafter “C5 building blocks”) wherein the methods are biocatalyst based.

However, wild-type prokaryotes or eukaryotes do not overproduce such C5 building blocks to the extracellular environment. Nevertheless, the metabolism of glutaric acid, 5-aminopentanoate and cadaverine has been reported.

The dicarboxylic acid glutaric acid is converted efficiently as a carbon source by a number of bacteria and yeasts via β-oxidation into central metabolites. Decarboxylation of Coenzyme A (CoA) activated glutarate to crotonyl-CoA facilitates further catabolism via β-oxidation.

The metabolism of 5-aminopentanoate has been reported for anaerobic bacteria such as Clostridium viride (Buckel et al., 2004, Arch. Microbiol., 162, 387-394). Similarly, cadaverine may be degraded to acetate and butyrate (Roeder and Schink, 2009, Appl. Environ. Microbiol., 75(14), 4821-4828)

The optimality principle states that microorganisms regulate their biochemical networks to support maximum biomass growth. Beyond the need for expressing heterologous pathways in a host organism, directing carbon flux towards C5 building blocks that serve as carbon sources rather than as biomass growth constituents, contradicts the optimality principle. For example, transferring the 1-butanol pathway from Clostridium species into other production strains has often fallen short by an order of magnitude compared to the production performance of native producers (Shen et al., Appl. Environ. Microbiol., 2011, 77(9):2905-2915).

The efficient synthesis of the five carbon aliphatic backbone precursor is a key consideration in synthesizing one or more C5 building blocks prior to forming terminal functional groups, such as carboxyl, amine or hydroxyl groups, on the C5 aliphatic backbone.

### SUMMARY

This document is based at least in part on the discovery that it is possible to construct biochemical pathways for producing a five carbon chain backbone precursor such as L-lysine, in which one or two functional groups, i.e., carboxyl, amine or hydroxyl, can be formed, leading to the synthesis of one or more of glutaric acid, 5-hydroxypentanoate, 5-aminopentanoate, cadaverine (also known as 1,5 pentanediamine), and 1,5-pentanediol (hereafter “C5 building blocks). Glutarate semialdehyde (also known as 5-oxopentanoic acid) can be produced as an intermediate to other products. Glutaric acid and glutarate, 5-hydroxypentanoic acid and 5-hydroxypentanoate, 5-oxopentanoic acid and 5-oxopentanoate, and 5-aminopentanoic and 5-aminopentanoate are used interchangeably herein to refer to the compound in any of its neutral or ionized forms, including any salt forms thereof. It is understood by those skilled in the art that the specific form will depend on pH.

In some embodiments, the C5 aliphatic backbone for conversion to a C5 building block can be formed from 2-oxoglutarate or oxaloacetate via conversion to L-lysine, followed by (i) decarboxylation to cadaverine, or (ii) conversion by monooxygenase activity to 5-aminopentanamide. See, FIGS. 1 to 3.

In some embodiments, an enzyme in the pathway generating the C5 aliphatic backbone purposefully contains irreversible enzymatic steps.

In some embodiments, the terminal carboxyl groups can be enzymatically formed using (i) an amidase such as 5-aminopentanamidase, (ii) an oxidase such as a primary-amine oxidase, (iii) an aldehyde dehydrogenase, such as a 7-oxoheptanoate dehydrogenase, a 6-oxohexanoate dehydrogenase or a 5-oxopentanoate dehydrogenase. See, FIG. 4-6.

In some embodiments, the terminal amine groups can be enzymatically formed using a decarboxylase such as a lysine decarboxylase, an ornithine decarboxylase, a glutamate decarboxylase or an arginine decarboxylase. See, FIG. 2.

In some embodiments, the terminal hydroxyl group can be enzymatically formed using an alcohol dehydrogenase such as a 4-hydroxybutyrate dehydrogenase, a 5-hydroxypentanoate dehydrogenase, or a 6-hydroxyhexanoate dehydrogenase. See, FIGS. 8 and 9.

A ω-transaminase can have at least 70% sequence identity to any one of the amino acid sequences set forth in SEQ ID NOs: 8 to 13.

A carboxylate reductase (e.g., in combination with a phosphopantetheinyl transferase) can form a terminal aldehyde group as an intermediate in forming the product. The carboxylate reductase can have at least 70% sequence identity to any one of the amino acid sequences set forth in SEQ ID NOs: 2 to 7.

A decarboxylase can have at least 70% sequence identity to any one of the amino acid sequences set forth in SEQ ID NOs: 1 and 16 to 18.

Any of the methods can be performed in a recombinant host by fermentation. The host can be subjected to a cultivation strategy under aerobic, anaerobic, or micro-aerobic cultivation conditions. The host can be cultured under conditions of nutrient limitation such as phosphate, oxygen or nitrogen limitation. The host can be retained using a ceramic membrane to maintain a high cell density during fermentation.

In any of the methods, the host's tolerance to high concentrations of a C5 building block can be improved through continuous cultivation in a selective environment.

The principal carbon source fed to the fermentation can derive from biological or non-biological feedstocks. In some embodiments, the biological feedstock is, includes, or derives from, monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

In some embodiments, the non-biological feedstock is or derives from natural gas, syngas, CO₂/H₂, methanol, ethanol, benzoate, non-volatile residue (NVR) or a caustic wash waste stream from cyclohexane oxidation processes, or a terephthalic acid/isophthalic acid mixture waste stream.

This document also features a recombinant host that includes at least one exogenous nucleic acid encoding a (i) decarboxylase and (ii) an oxidase, and produce cadaverine or 5-aminopentanoate.

This document further features a recombinant host that includes at least one exogenous nucleic acid encoding (i) a lysine 2-monooxygenase and (ii) a 5-aminopentanamidase, and produces 5-aminopentanoate.

Such a recombinant host producing 5-aminopentanoate further can include one or more of (i) a ω-transaminase or (ii) an aldehyde dehydrogenase, 7-oxoheptanoate dehydrogenase, 6-oxohexanoate dehydrogenase, or 5-oxopentanoate dehydrogenase and further produce glutarate semialdehyde and glutaric acid. Also, such a recombinant host producing 5-aminopentanoate further can include one or more of (i) a ω-transaminase or (ii) an alcohol dehydrogenase, 4-hydroxybutyrate dehydrogenase, 5-hydroxypentanoate dehydrogenase or 6-hydroxyhexanoate dehydrogenase and further produce 5-hydroxypentanoate.

A recombinant host producing 5-hydroxypentanoate can further include one or more of (i) a carboxylase reductase and (ii) an alcohol dehydrogenase, the host further producing 1,5-pentanediol.

The recombinant host can be a prokaryote, e.g., from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia acidovorans, from the genus Bacillus such as Bacillus subtillis; from the genes Lactobacillus such as Lactobacillus delbrueckii; from the genus Lactococcus such as Lactococcus lactis or from the genus Rhodococcus such as Rhodococcus equi.

The recombinant host can be a eukaryote, e.g., a eukaryote from the genus Aspergillus such as Aspergillus niger; from the genus Saccharomyces such as Saccharomyces cerevisiae; from the genus Pichia such as Pichia pastoris; from the genus Yarrowia such as Yarrowia lipolytica, from the genus Issatchenkia such as Issathenkia orientalis, from the genus Debaryomyces such as Debaryomyces hansenii, from the genus Arxula such as Arxula adenoinivorans, or from the genus Kluyveromyces such as Kluyveromyces lactis.

In some embodiments, the host's endogenous biochemical network is attenuated or augmented to (1) ensure the intracellular availability of 2-oxoglutarate or oxaloacetate, (2) create a NADPH cofactor imbalance that may be balanced via the formation of C5 building blocks, (3) prevent degradation of central metabolites, central precursors leading to and including C5 building blocks and (4) ensure efficient efflux from the cell.

Any of the recombinant hosts described herein further can include one or more of the following attenuated enzymes: polyhydroxyalkanoate synthase, an acetyl-CoA thioesterase, an acetyl-CoA specific β-ketothiolase, an acetoacetyl-CoA reductase, a phosphotransacetylase forming acetate, an acetate kinase, a lactate dehydrogenase, a menaquinol-fumarate oxidoreductase, a 2-oxoacid decarboxylase producing isobutanol, an alcohol dehydrogenase forming ethanol, a triose phosphate isomerase, a pyruvate decarboxylase, a glucose-6-phosphate isomerase, a transhydrogenase dissipating the cofactor imbalance, an NADH-specific glutamate dehydrogenase, a NADH/NADPH-utilizing glutamate dehydrogenase, a glutaryl-CoA dehydrogenase, or an acyl-CoA dehydrogenase accepting C5 building blocks and central precursors as substrates.

Any of the recombinant hosts described herein further can overexpress one or more genes encoding: an acetyl-CoA synthetase, a 6-phosphogluconate dehydrogenase; a transketolase; a puridine nucleotide transhydrogenase; a formate dehydrogenase; a glyceraldehyde-3P-dehydrogenase; a malic enzyme; a glucose-6-phosphate dehydrogenase; a fructose 1,6 diphosphatase; a propionyl-CoA synthetase; a L-alanine dehydrogenase; an NADPH-specific L-glutamate dehydrogenase; a PEP carboxylase, a pyruvate carboxylase, PEP carboxykinase, PEP synthase, a L-glutamine synthetase; a lysine transporter; a dicarboxylate transporter; and/or a multidrug transporter.

The reactions of the pathways described herein can be performed in one or more cell (e.g., host cell) strains (a) naturally expressing one or more relevant enzymes, (b) genetically engineered to express one or more relevant enzymes, or (c) naturally expressing one or more relevant enzymes and genetically engineered to express one or more relevant enzymes. Alternatively, relevant enzymes can be extracted from of the above types of host cells and used in a purified or semi-purified form. Extracted enzymes can optionally be immobilized to the floors and/or walls of appropriate reaction vessels. Moreover, such extracts include lysates (e.g., cell lysates) that can be used as sources of relevant enzymes. In the methods provided by the document, all the steps can be performed in cells (e.g., host cells), all the steps can be performed using extracted enzymes, or some of the steps can be performed in cells and others can be performed using extracted enzymes.

Many of the enzymes described herein catalyze reversible reactions, and the reaction of interest may be the reverse of the described reaction. The schematic pathways shown in FIGS. 1 to 9 illustrate the reaction of interest for each of the intermediates.

In one aspect, this document features a method for producing a bioderived five carbon compound. The method for producing a bioderived five carbon compound can include culturing or growing a recombinant host as described herein under conditions and for a sufficient period of time to produce the bioderived five carbon compound, wherein, optionally, the bioderived five carbon compound is selected from the group consisting of glutaric acid, 5 aminopentanoic acid, 5-hydroxypentanoic acid, cadaverine, 1,5-pentanediol, and combinations thereof.

In one aspect, this document features composition comprising a bioderived five carbon compound as described herein and a compound other than the bioderived five carbon compound, wherein the bioderived 5-carbon compound is selected from the group consisting of glutaric acid, 5-aminopentanoic acid, 5-hydroxypentanoic acid, cadaverine, 1,5-pentanediol, and combinations thereof. For example, the bioderived five carbon compound is a cellular portion of a host cell or an organism.

This document also features a biobased polymer comprising the bioderived glutaric acid, 5-aminopentanoic acid, 5-hydroxypentanoic acid, cadaverine, 1,5-pentanediol, and combinations thereof.

This document also features a biobased resin comprising the bioderived glutaric acid, 5-aminopentanoic acid, 5-hydroxypentanoic acid, cadaverine, 1,5-pentanediol, and combinations thereof, as well as a molded product obtained by molding a biobased resin.

In another aspect, this document features a process for producing a biobased polymer that includes chemically reacting the bioderived glutaric acid, 5-aminopentanoic acid, 5-hydroxypentanoic acid, cadaverine, or 1,5-pentanediol, with itself or another compound in a polymer producing reaction.

In another aspect, this document features a process for producing a biobased resin that includes chemically reacting the bioderived glutaric acid, 5 aminopentanoic acid, 5-hydroxypentanoic acid, cadaverine, or 1,5-pentanediol, with itself or another compound in a resin producing reaction.

Also described herein is a biochemical network comprising a polypeptide having decarboxylase activity to enzymatically convert lysine to cadaverine; and a polypeptide having oxidase activity to enzymatically convert cadaverine to 5-aminopentanoate. A biochemical network comprising a polypeptide having monooxygenase activity to enzymatically convert lysine to 5-aminopentanamide; and a polypeptide having amidase activity to enzymatically convert 5-aminopentanamide to 5-aminopentanoate is also provided. Also described herein is a biochemical network comprising a polypeptide having ω-transaminase activity to enzymatically convert cadaverine to 5-aminopentanal; and a polypeptide having aldehyde dehydrogenase activity to enzymatically convert 5-aminopentanal to 5-aminopentanoate. A biochemical network can further include a polypeptide having decarboxylase activity to enzymatically convert lysine to cadaverine.

The biochemical network can further include one or more polypeptides having transaminase, dehydrogenase, or carboxylate reductase activity, wherein the one or more polypeptides having transaminase, dehydrogenase, or carboxylate reductase activity enzymatically convert 5-aminopentanoate to a product selected from the group consisting of glutaric acid, 5 aminopentanoic acid, 5-hydroxypentanoic acid, cadaverine, and 1,5-pentanediol.

Also described herein is a means for obtaining glutaric acid, 5 aminopentanoic acid, 5-hydroxypentanoic acid, cadaverine, and 1,5-pentanediol using one or more polypeptides having transaminase, dehydrogenase, or carboxylate reductase activity.

In another aspect, this document features a composition comprising one or more polypeptides having transaminase, dehydrogenase, or carboxylate reductase activity and at least one of glutaric acid, 5 aminopentanoic acid, 5-hydroxypentanoic acid, cadaverine, and 1,5-pentanediol. The composition can be cellular.

In another aspect, this document features a bio-derived product, a bio-based product or a fermentation-derived product, the product comprising i. a composition comprising at least one bio-derived, bio-based or fermentation-derived compound according to any one of claims 1-53, or any one of FIGS. 1-16, or any combination thereof, ii. a bio-derived, bio-based or fermentation-derived polymer comprising the bio-derived, bio-based or fermentation-derived composition or compound of i., or any combination thereof, iii. a bio-derived, bio-based or fermentation-derived resin comprising the bio-derived, bio-based or fermentation-derived compound or bio-derived, bio-based or fermentation-derived composition of i. or any combination thereof or the bio-derived, bio-based or fermentation-derived polymer of ii. or any combination thereof, iv. a molded substance obtained by molding the bio-derived, bio-based or fermentation-derived polymer of ii. or the bio-derived, bio-based or fermentation-derived resin of iii., or any combination thereof, v. a bio-derived, bio-based or fermentation-derived formulation comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., or bio-derived, bio-based or fermentation-derived molded substance of iv, or any combination thereof, or vi. a bio-derived, bio-based or fermentation-derived semi-solid or a non-semi-solid stream, comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., bio-derived, bio-based or fermentation-derived formulation of v., or bio-derived, bio-based or fermentation-derived molded substance of iv., or any combination thereof.

One of skill in the art understands that compounds containing carboxylic acid groups (including, but not limited to, organic monoacids, hydroxyacids, aminoacids, and dicarboxylic acids) are formed or converted to their ionic salt form when an acidic proton present in the parent compound either is replaced by a metal ion, e.g., an alkali metal ion, an alkaline earth ion, or an aluminum ion; or coordinates with an organic base. Acceptable organic bases include, but are not limited to, ethanolamine, diethanolamine, triethanolamine, tromethamine, N-methylglucamine, and the like. Acceptable inorganic bases include, but are not limited to, aluminum hydroxide, calcium hydroxide, potassium hydroxide, sodium carbonate, sodium hydroxide, and the like. A salt of the present invention is isolated as a salt or converted to the free acid by reducing the pH to below the pKa, through addition of acid or treatment with an acidic ion exchange resin.

One of skill in the art understands that compounds containing amine groups (including, but not limited to, organic amines, aminoacids, and diamines) are formed or converted to their ionic salt form, for example, by addition of an acidic proton to the amine to form the ammonium salt, formed with inorganic acids such as hydrochloric acid, hydrobromic acid, sulfuric acid, nitric acid, phosphoric acid, and the like; or formed with organic acids including, but not limited to, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include, but are not limited to, aluminum hydroxide, calcium hydroxide, potassium hydroxide, sodium carbonate, sodium hydroxide, and the like. A salt of the present invention is isolated as a salt or converted to the free amine by raising the pH to above the pKb through addition of base or treatment with a basic ion exchange resin.

One of skill in the art understands that compounds containing both amine groups and carboxylic acid groups (including, but not limited to, aminoacids) are formed or converted to their ionic salt form by either 1) acid addition salts, formed with inorganic acids including, but not limited to, hydrochloric acid, hydrobromic acid, sulfuric acid, nitric acid, phosphoric acid, and the like; or formed with organic acids including, but not limited to, acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethanedisulfonic acid, 2-hydroxyethanesulfonic acid, benzenesulfonic acid, 2-naphthalenesulfonic acid, 4-methylbicyclo-[2.2.2]oct-2-ene-1-carboxylic acid, glucoheptonic acid, 4,4′-methylenebis-(3-hydroxy-2-ene-1-carboxylic acid), 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. Acceptable inorganic bases include, but are not limited to, aluminum hydroxide, calcium hydroxide, potassium hydroxide, sodium carbonate, sodium hydroxide, and the like, or 2) when an acidic proton present in the parent compound either is replaced by a metal ion, e.g., an alkali metal ion, an alkaline earth ion, or an aluminum ion; or coordinates with an organic base. Acceptable organic bases include, but are not limited to, ethanolamine, diethanolamine, triethanolamine, tromethamine, N-methylglucamine, and the like. Acceptable inorganic bases include, but are not limited to, aluminum hydroxide, calcium hydroxide, potassium hydroxide, sodium carbonate, sodium hydroxide, and the like. A salt can of the present invention is isolated as a salt or converted to the free acid by reducing the pH to below the pKa through addition of acid or treatment with an acidic ion exchange resin.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein including GenBank and NCBI submissions with accession numbers are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and the drawings, and from the claims. The word “comprising” in the claims may be replaced by “consisting essentially of” or with “consisting of,” according to standard practice in patent law.

### DESCRIPTION OF DRAWINGS

FIG. 1 is a schematic of exemplary biochemical pathways leading to L-lysine using 2-oxoglutarate or oxaloacetate as a central metabolite.

FIG. 2 is a schematic of an exemplary biochemical pathway leading to cadaverine using L-lysine as a central metabolite.

FIG. 3 is a schematic of an exemplary biochemical pathway leading to the C5 carbon backbone 5-aminopentanamide using L-lysine as a central metabolite.

FIG. 4 is a schematic of an exemplary biochemical pathway leading to 5-aminopentanoate (also known as 5-aminovalerate) using 5-aminopentanamide as central precursor.

FIG. 5 is a schematic of an exemplary biochemical pathway leading to 5-aminopentanoate using cadaverine as a central precursor.

FIG. 6 is a schematic of an exemplary biochemical pathway leading to 5-aminopentanoate using cadaverine as a central precursor.

FIG. 7 is a schematic of an exemplary biochemical pathway leading to glutarate using 5-aminopentanoate as a central precursor.

FIG. 8 is a schematic of an exemplary biochemical pathway leading to 5-hydroxypentanoate using 5-aminopentanoate as a central precursor.

FIG. 9 is a schematic of an exemplary biochemical pathway leading to 1,5 pentanediol using 5-hydroxypentanoate as a central precursor.

FIG. 10 contains the amino acid sequences of an Escherichia coli lysine decarboxylase (see Genbank Accession No. BAA21656.1, SEQ ID NO: 1), a Mycobacterium marinum carboxylate reductase (see Genbank Accession No. ACC40567.1, SEQ ID NO: 2), a Mycobacterium smegmatis carboxylate reductase (see Genbank Accession No. ABK71854.1, SEQ ID NO: 3), a Segniliparus rugosus carboxylate reductase (see Genbank Accession No. EFV11917.1, SEQ ID NO: 4), a Mycobacterium smegmatis carboxylate reductase (see Genbank Accession No. ABK75684.1, SEQ ID NO: 5), a Mycobacterium massiliense carboxylate reductase (see Genbank Accession No. EIV11143.1, SEQ ID NO: 6), a Segniliparus rotundus carboxylate reductase (see Genbank Accession No. ADG98140.1, SEQ ID NO: 7), a Chromobacterium violaceum ω-transaminase (see Genbank Accession No. AAQ59697.1, SEQ ID NO: 8), a Pseudomonas aeruginosa ω-transaminase (see Genbank Accession No. AAG08191.1, SEQ ID NO: 9), a Pseudomonas syringae ω-transaminase (see Genbank Accession No. AAY39893.1, SEQ ID NO: 10), a Rhodobacter sphaeroides ω-transaminase (see Genbank Accession No. ABA81135.1, SEQ ID NO: 11), an Escherichia coli ω-transaminase (see Genbank Accession No. AAA57874.1, SEQ ID NO: 12), a Vibrio fluvialis ω-transaminase (See Genbank Accession No. AEA39183.1, SEQ ID NO: 13), a Bacillus subtilis phosphopantetheinyl transferase (see Genbank Accession No. CAA44858.1, SEQ ID NO: 14), a Nocardia sp. NRRL 5646 phosphopantetheinyl transferase (see Genbank Accession No. ABI83656.1, SEQ ID NO: 15), an Escherichia coli glutamate decarboxylase (see Genbank Accession No. AAA23833.1, SEQ ID NO: 16), an Escherichia coli lysine decarboxylase (see Genbank Accession No. AAA23536.1, SEQ ID NO: 17), an Escherichia coli ornithine decarboxylase (see Genbank Accession No. AAA23536.1, SEQ ID NO: 18), a Pseudomonas putida 5-aminopentanamidase (see Genbank Accession No. ADI95308.1, SEQ ID NO: 19), a Pseudomonas putida lysine-2-monooxygenase (see Genbank Accession No. BAG54787.1, SEQ ID NO: 20), and an Escherichia coli primary amine oxidase (see Genbank Accession No. BAA04900.1, SEQ ID NO: 21).

FIG. 11 is a bar graph of the percent conversion after 4 hours of pyruvate to L-alanine (mol/mol) as a measure of the ω-transaminase activity of four ω-transaminase preparations for converting cadaverine to 5-aminopentanal relative to the empty vector control.

FIG. 12 is a bar graph summarizing the change in absorbance at 340 nm after 20 minutes, which is a measure of the consumption of NADPH and activity of five carboxylate reductase preparations in enzyme only controls (no substrate).

FIG. 13 is a bar graph summarizing the percent conversion of pyruvate to L-alanine (mol/mol) as a measure of the ω-transaminase activity of the enzyme only controls (no substrate).

FIG. 14 is a bar graph of the change in absorbance at 340 nm after 20 minutes, which is a measure of the consumption of NADPH and the activity of five carboxylate reductase preparations for converting 5-hydroxypentanoate to 5-hydroxypentanal relative to the empty vector control.

FIG. 15 is a bar graph of the percent conversion after 4 hours of pyruvate to L-alanine (mol/mol) as a measure of the ω-transaminase activity of one ω-transaminase preparation for converting 5-aminopentanoate to glutarate semialdehyde relative to the empty vector control.

FIG. 16 is a bar graph of the percent conversion after 4 hours of L-alanine to pyruvate (mol/mol) as a measure of the ω-transaminase activity of one ω-transaminase preparations for converting glutarate semialdehyde to 5-aminopentanoate relative to the empty vector control.

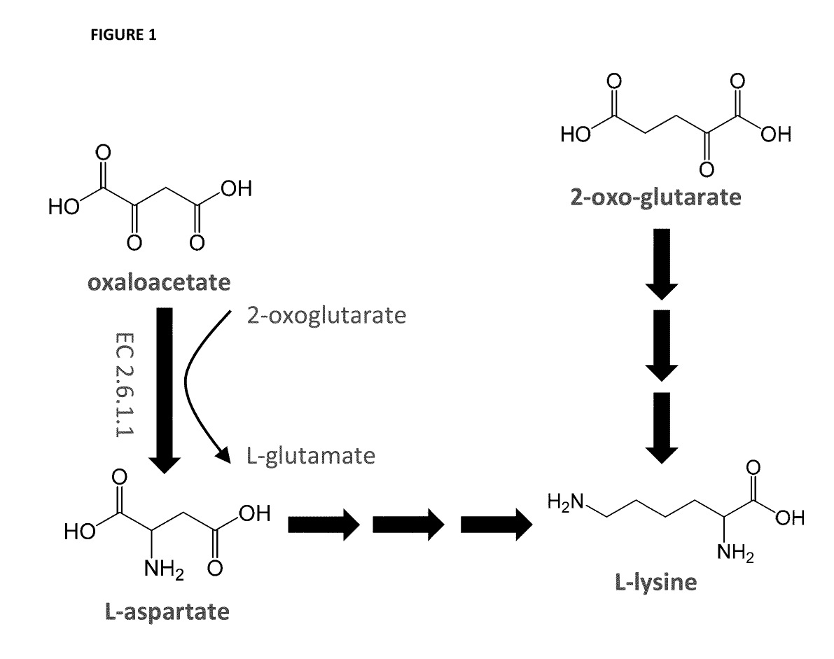

### DETAILED DESCRIPTION

This document provides enzymes, non-natural pathways, cultivation strategies, feedstocks, host microorganisms and attenuations to the host's biochemical network, which generates a five carbon chain backbone such as cadaverine or 5-aminopentanamide from central metabolites in which one or two terminal functional groups may be formed leading to the synthesis of one or more of glutaric acid, 5-aminopentanoic acid, cadaverine (also known as 1,5 pentanediamine), 5-hydroxypentanoic acid, or 1,5-pentanediol (hereafter “C5 building blocks”). Glutarate semialdehyde (also known as 5-oxopentanoate) can be produced as an intermediate to other products. As used herein, the term “central precursor” is used to denote any metabolite in any metabolic pathway shown herein leading to the synthesis of a C5 building block. The term “central metabolite” is used herein to denote a metabolite that is produced in all microorganisms to support growth.

Host microorganisms described herein can include endogenous pathways that can be manipulated such that one or more C5 building blocks can be produced. In an endogenous pathway, the host microorganism naturally expresses all of the enzymes catalyzing the reactions within the pathway. A host microorganism containing an engineered pathway does not naturally express all of the enzymes catalyzing the reactions within the pathway but has been engineered such that all of the enzymes within the pathway are expressed in the host.

The term “exogenous” as used herein with reference to a nucleic acid (or a protein) and a host refers to a nucleic acid that does not occur in (and cannot be obtained from) a cell of that particular type as it is found in nature or a protein encoded by such a nucleic acid. Thus, a non-naturally-occurring nucleic acid is considered to be exogenous to a host once in the host. It is important to note that non-naturally-occurring nucleic acids can contain nucleic acid subsequences or fragments of nucleic acid sequences that are found in nature provided the nucleic acid as a whole does not exist in nature. For example, a nucleic acid molecule containing a genomic DNA sequence within an expression vector is non-naturally-occurring nucleic acid, and thus is exogenous to a host cell once introduced into the host, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that genomic DNA fragments produced by PCR or restriction endonuclease treatment as well as cDNAs are considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. A nucleic acid that is naturally-occurring can be exogenous to a particular host microorganism. For example, an entire chromosome isolated from a cell of yeast x is an exogenous nucleic acid with respect to a cell of yeast y once that chromosome is introduced into a cell of yeast y.

In contrast, the term “endogenous” as used herein with reference to a nucleic acid (e.g., a gene) (or a protein) and a host refers to a nucleic acid (or protein) that does occur in (and can be obtained from) that particular host as it is found in nature. Moreover, a cell “endogenously expressing” a nucleic acid (or protein) expresses that nucleic acid (or protein) as does a host of the same particular type as it is found in nature. Moreover, a host “endogenously producing” or that “endogenously produces” a nucleic acid, protein, or other compound produces that nucleic acid, protein, or compound as does a host of the same particular type as it is found in nature.

For example, depending on the host and the compounds produced by the host, one or more of the following polypeptides may be expressed in the host including a polypeptide having decarboxylase activity such as a lysine decarboxylase, an ornithine decarboxylase, a glutamate decarboxylase or an arginine decarboxylase, a lysine 2-monooxygenase, a 5-aminopentanamidase, a primary-amine oxidase, 4-hydroxybutyrate dehydrogenase, a 5-hydroxypentanoate dehydrogenase, a 6-hydroxyhexanoate dehydrogenase, an alcohol dehydrogenase, a 5-oxopentanoate dehydrogenase, a 6-oxohexanoate dehydrogenase, a 7-oxoheptanoate dehydrogenase, an aldehyde dehydrogenase, a ω-transaminase, or a carboxylate reductase. In recombinant hosts expressing a carboxylate reductase, a phosphopantetheinyl transferase also can be expressed as it enhances activity of the carboxylate reductase.

In some embodiments, a recombinant host that produces L-lysine can include at least one exogenous nucleic acid encoding (i) a decarboxylase such as a lysine decarboxylase, an ornithine decarboxylase, a glutamate decarboxylase or an arginine decarboxylase and (ii) a primary-amine oxidase, and further produce cadaverine or 5-aminopentanoate.

In some embodiments, a recombinant host that produces L-lysine can include at least one exogenous nucleic acid encoding (i) a lysine-2-monooxygenase and (ii) a 5-aminopentanamidase, and further produce 5-aminopentanoate.

In some embodiments, a recombinant host producing 5-aminopentanoate includes at least one exogenous nucleic acid encoding (i) a reversible ω-transaminase (e.g., a 5-aminovalerate transaminase) and (ii) an aldehyde dehydrogenase such as a succinate semialdehyde dehydrogenase, a 5-oxovalerate dehydrogenase, a 6-oxohexanoate dehydrogenase, or a 7-oxoheptanoate dehydrogenase and produces glutarate or glutarate semialdehyde. For example, a host producing 5-aminopentanoate can include a reversible ω-transaminase (e.g., a 5-aminovalerate transaminase) and produce glutarate semialdehyde. For example, a host producing 5-aminopentanoate can include (i) a reversible ω-transaminase (e.g., a 5-aminovalerate transaminase) and (ii) an aldehyde dehydrogenase such as a succinate semialdehyde dehydrogenase, a 5-oxovalerate dehydrogenase, a 6-oxohexanoate dehydrogenase, or a 7-oxoheptanoate dehydrogenase and produce glutarate.

In some embodiments, a recombinant host that produces 5-aminopentanoate can include at least one exogenous nucleic acid encoding (i) a reversible co transaminase (e.g., a 5-aminovalerate transaminase) and (ii) an alcohol dehydrogenase such as 4-hydroxybutyrate dehydrogenase, a 5-hydroxypentanoate dehydrogenase, a 6-hydroxyhexanoate dehydrogenase, and further produce 5-hydroxypentanoate.

A recombinant host producing 5-hydroxypentanoic acid further can include one or more of (i) a carboxylate reductase and (ii) an alcohol dehydrogenase, and produce 1,5-pentanediol.

Within an engineered pathway, the enzymes can be from a single source, i.e., from one species or genus, or can be from multiple sources, i.e., different species or genera. Nucleic acids encoding the enzymes described herein have been identified from various organisms and are readily available in publicly available databases such as GenBank or EMBL.

Any of the enzymes described herein that can be used for production of one or more C5 building blocks can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of the corresponding wild-type enzyme. It will be appreciated that the sequence identity can be determined on the basis of the mature enzyme (e.g., with any signal sequence removed) or on the basis of the immature enzyme (e.g., with any signal sequence included). It also will be appreciated that the initial methionine residue may or may not be present on any of the enzyme sequences described herein.

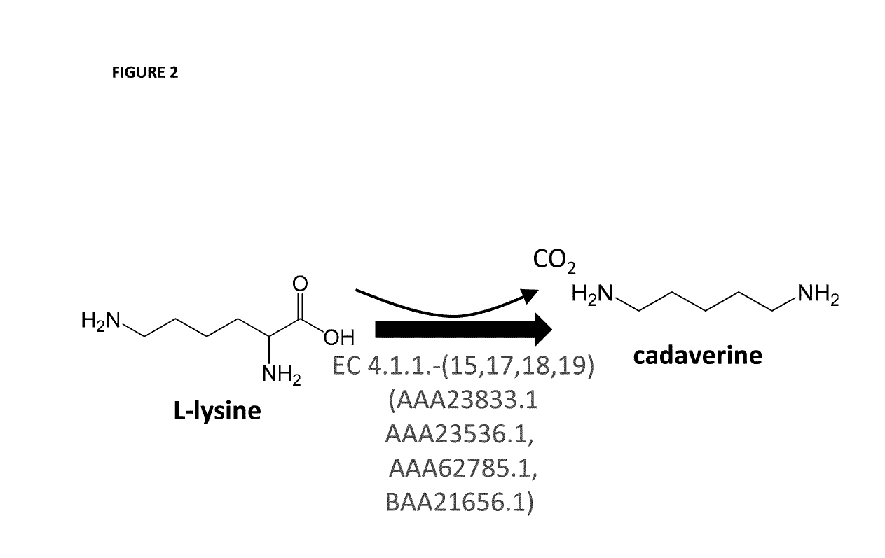

For example, a polypeptide having decarboxylase activity described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence from an Escherichia coli (see Genbank Accession Nos. AAA23833.1, AAA23536.1, AAA62785.1, BAA21656.1, SEQ ID NOs: 1 and 16-18). See, FIG. 2.

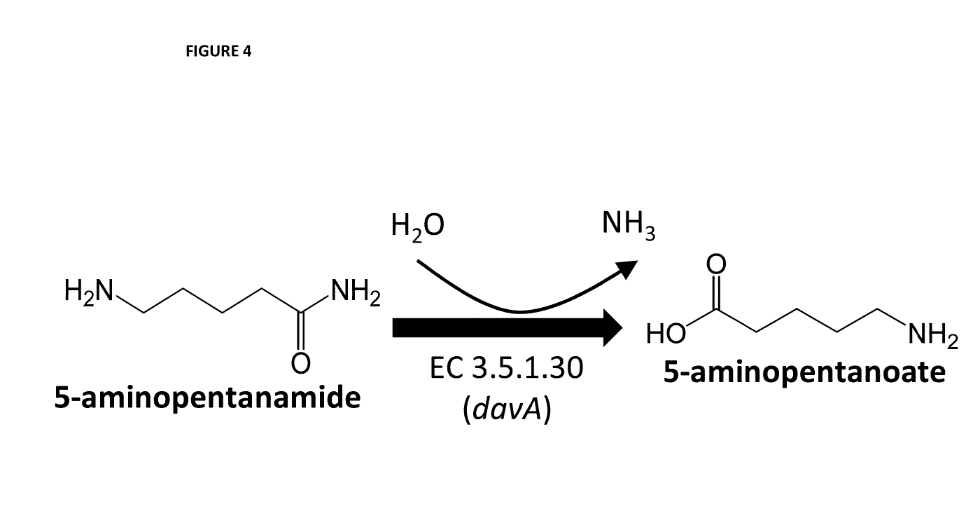

For example, a polypeptide having 5-aminopentanamidase activity described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Pseudomonas putida (see Genbank Accession No. ADI95308.1, SEQ ID NO: 19). See, FIG. 4.

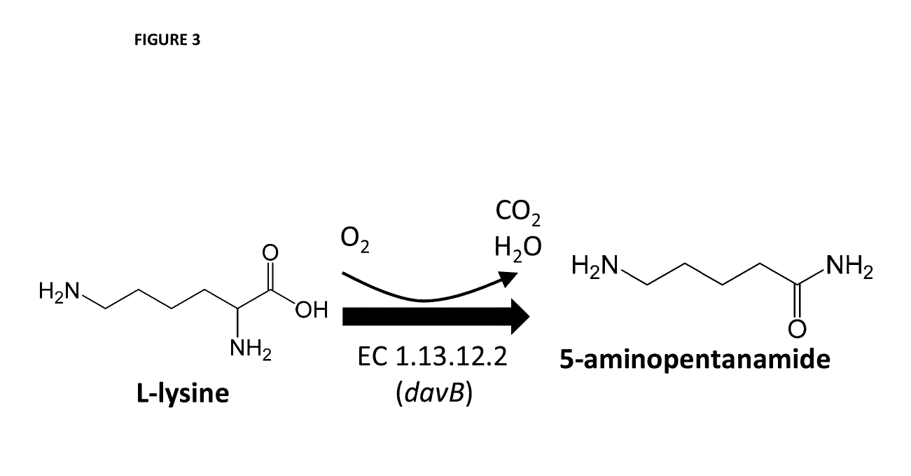

For example, a polypeptide having lysine-2-monooxygenase activity described herein can have at least 70% sequence identity (homology) (e.g., at least 75%, 80%, 85%, 90%, 95%, 97%, 98%, 99%, or 100%) to the amino acid sequence of a Pseudomonas putida (see Genbank Accession No. BAG54787.1, SEQ ID NO: 20). See, FIG. 3.

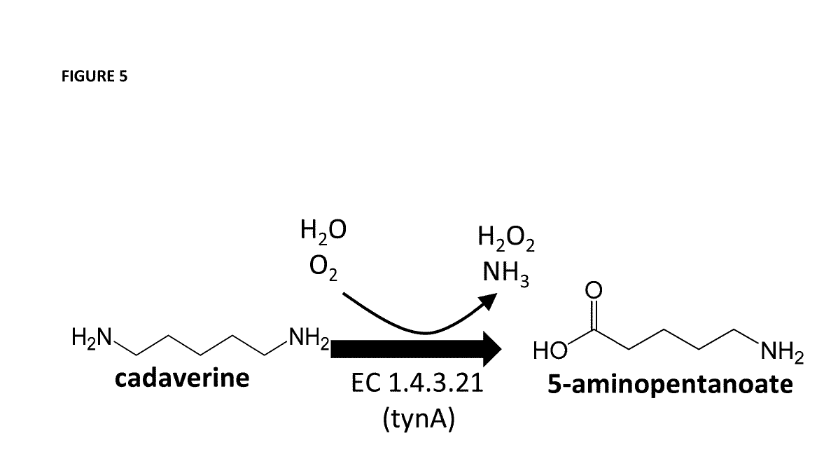

For example, a polypeptide having primary-amine oxidase activity described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of an Escherichia coli (see Genbank Accession No. BAA04900.1, SEQ ID NO: 21) primary-amine oxidase. See, FIG. 5.

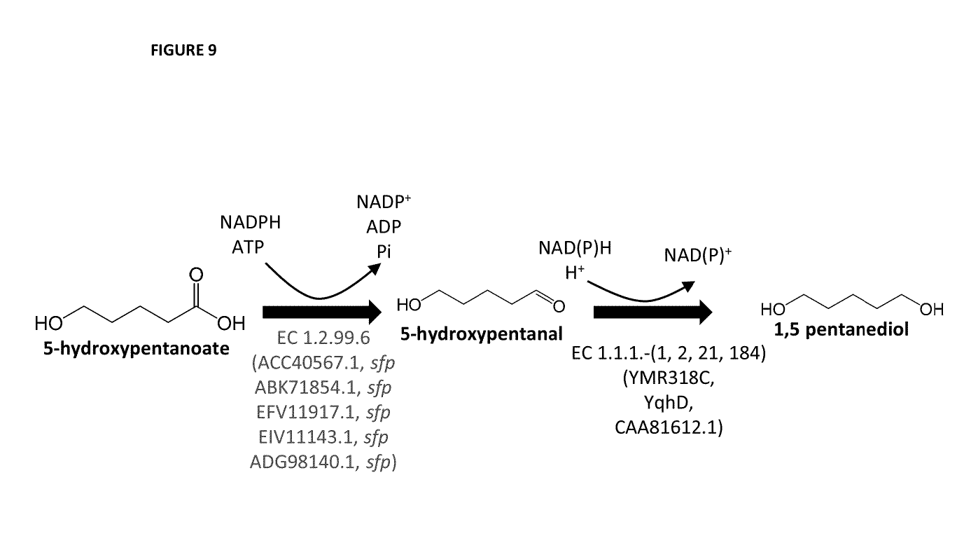

For example, a polypeptide having carboxylate reductase activity described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Mycobacterium marinum (see Genbank Accession No. ACC40567.1, SEQ ID NO: 2), a Mycobacterium smegmatis (see Genbank Accession No. ABK71854.1, SEQ ID NO: 3), a Segniliparus rugosus (see Genbank Accession No. EFV11917.1, SEQ ID NO: 4), a Mycobacterium smegmatis (see Genbank Accession No. ABK75684.1, SEQ ID NO: 5), a Mycobacterium massiliense (see Genbank Accession No. EIV11143.1, SEQ ID NO: 6), or a Segniliparus rotundus (see Genbank Accession No. ADG98140.1, SEQ ID NO: 7) carboxylate reductase. See, FIG. 9.

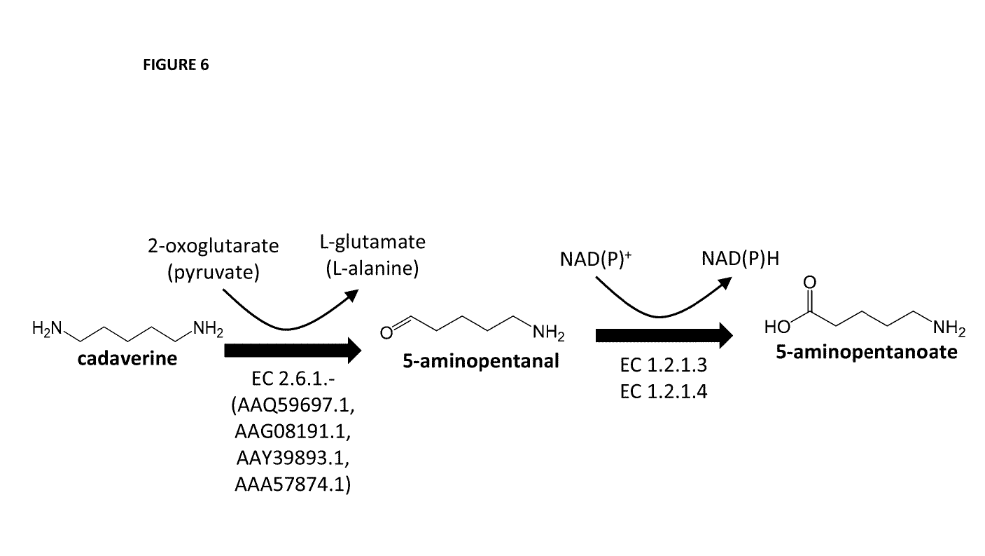

For example, a polypeptide having ω-transaminase activity described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Chromobacterium violaceum (see Genbank Accession No. AAQ59697.1, SEQ ID NO: 8), a Pseudomonas aeruginosa (see Genbank Accession No. AAG08191.1, SEQ ID NO: 9), a Pseudomonas syringae (see Genbank Accession No. AAY39893.1, SEQ ID NO: 10), a Rhodobacter sphaeroides (see Genbank Accession No. ABA81135.1, SEQ ID NO: 11), an Escherichia coli (see Genbank Accession No. AAA57874.1, SEQ ID NO: 12), or a Vibrio fluvialis (see Genbank Accession No. AEA39183.1, SEQ ID NO: 13) ω-transaminase. Some of these ω-transaminases are diamine ω-transaminases. See, FIGS. 6-8.

For example, a polypeptide having phosphopantetheinyl transferase activity described herein can have at least 70% sequence identity (homology) (e.g., at least 75%, 80%, 85%, 90%, 91%, 92%, 93%, 94%, 95%, 96%, 97%, 98%, 99%, or 100%) to the amino acid sequence of a Bacillus subtilis phosphopantetheinyl transferase (see Genbank Accession No. CAA44858.1, SEQ ID NO: 14) or a Nocardia sp. NRRL 5646 phosphopantetheinyl transferase (see Genbank Accession No. ABI83656.1, SEQ ID NO: 15). See, FIG. 9.

The percent identity (homology) between two amino acid sequences can be determined as follows. First, the amino acid sequences are aligned using the BLAST 2 Sequences (Bl2seq) program from the stand-alone version of BLASTZ containing BLASTP version 2.0.14. This stand-alone version of BLASTZ can be obtained from Fish & Richardson's web site (e.g., www.fr.com/blast/) or the U.S. government's National Center for Biotechnology Information web site (www.ncbi.nlm.nih.gov). Instructions explaining how to use the Bl2seq program can be found in the readme file accompanying BLASTZ. Bl2seq performs a comparison between two amino acid sequences using the BLASTP algorithm. To compare two amino acid sequences, the options of Bl2seq are set as follows: -i is set to a file containing the first amino acid sequence to be compared (e.g., C:\seq1.txt); -j is set to a file containing the second amino acid sequence to be compared (e.g., C:\seq2.txt); -p is set to blastp; -o is set to any desired file name (e.g., C:\output.txt); and all other options are left at their default setting. For example, the following command can be used to generate an output file containing a comparison between two amino acid sequences: C:\Bl2seq-i c:\seq1.txt-j c:\seq2.txt-p blastp-o c:\output.txt. If the two compared sequences share homology (identity), then the designated output file will present those regions of homology as aligned sequences. If the two compared sequences do not share homology (identity), then the designated output file will not present aligned sequences. Similar procedures can be following for nucleic acid sequences except that blastn is used.

Once aligned, the number of matches is determined by counting the number of positions where an identical amino acid residue is presented in both sequences. The percent identity (homology) is determined by dividing the number of matches by the length of the full-length polypeptide amino acid sequence followed by multiplying the resulting value by 100. It is noted that the percent identity (homology) value is rounded to the nearest tenth. For example, 78.11, 78.12, 78.13, and 78.14 is rounded down to 78.1, while 78.15, 78.16, 78.17, 78.18, and 78.19 is rounded up to 78.2. It also is noted that the length value will always be an integer.

It will be appreciated that a number of nucleic acids can encode a polypeptide having a particular amino acid sequence. The degeneracy of the genetic code is well known to the art; i.e., for many amino acids, there is more than one nucleotide triplet that serves as the codon for the amino acid. For example, codons in the coding sequence for a given enzyme can be modified such that optimal expression in a particular species (e.g., bacteria or fungus) is obtained, using appropriate codon bias tables for that species.

Functional fragments of any of the enzymes described herein can also be used in the methods of the document. The term “functional fragment” as used herein refers to a peptide fragment of a protein that has at least 25% (e.g., at least: 30%; 40%; 50%; 60%; 70%; 75%; 80%; 85%; 90%; 91%; 92%; 93%; 94%; 95%; 96%; 97%; 98%; 99%; 100%; or even greater than 100%) of the activity of the corresponding mature, full-length, wild-type protein. The functional fragment can generally, but not always, be comprised of a continuous region of the protein, wherein the region has functional activity.

This document also provides (i) functional variants of the enzymes used in the methods of the document and (ii) functional variants of the functional fragments described above. Functional variants of the enzymes and functional fragments can contain additions, deletions, or substitutions relative to the corresponding wild-type sequences. Enzymes with substitutions will generally have not more than 50 (e.g., not more than one, two, three, four, five, six, seven, eight, nine, ten, 12, 15, 20, 25, 30, 35, 40, or 50) amino acid substitutions (e.g., conservative substitutions). This applies to any of the enzymes described herein and functional fragments. A conservative substitution is a substitution of one amino acid for another with similar characteristics. Conservative substitutions include substitutions within the following groups: valine, alanine and glycine; leucine, valine, and isoleucine; aspartic acid and glutamic acid; asparagine and glutamine; serine, cysteine, and threonine; lysine and arginine; and phenylalanine and tyrosine. The nonpolar hydrophobic amino acids include alanine, leucine, isoleucine, valine, proline, phenylalanine, tryptophan and methionine. The polar neutral amino acids include glycine, serine, threonine, cysteine, tyrosine, asparagine and glutamine. The positively charged (basic) amino acids include arginine, lysine and histidine. The negatively charged (acidic) amino acids include aspartic acid and glutamic acid. Any substitution of one member of the above-mentioned polar, basic or acidic groups by another member of the same group can be deemed a conservative substitution. By contrast, a non-conservative substitution is a substitution of one amino acid for another with dissimilar characteristics.

Deletion variants can lack one, two, three, four, five, six, seven, eight, nine, ten, 11, 12, 13, 14, 15, 16, 17, 18, 19, or 20 amino acid segments (of two or more amino acids) or non-contiguous single amino acids. Additions (addition variants) include fusion proteins containing: (a) any of the enzymes described herein or a fragment thereof; and (b) internal or terminal (C or N) irrelevant or heterologous amino acid sequences. In the context of such fusion proteins, the term “heterologous amino acid sequences” refers to an amino acid sequence other than (a). A heterologous sequence can be, for example a sequence used for purification of the recombinant protein (e.g., FLAG, polyhistidine (e.g., hexahistidine), hemagglutinin (HA), glutathione-S-transferase (GST), or maltosebinding protein (MBP)). Heterologous sequences also can be proteins useful as detectable markers, for example, luciferase, green fluorescent protein (GFP), or chloramphenicol acetyl transferase (CAT). In some embodiments, the fusion protein contains a signal sequence from another protein. In certain host cells (e.g., yeast host cells), expression and/or secretion of the target protein can be increased through use of a heterologous signal sequence. In some embodiments, the fusion protein can contain a carrier (e.g., KLH) useful, e.g., in eliciting an immune response for antibody generation) or ER or Golgi apparatus retention signals. Heterologous sequences can be of varying length and in some cases can be a longer sequences than the full-length target proteins to which the heterologous sequences are attached.

Engineered hosts can naturally express none or some (e.g., one or more, two or more, three or more, four or more, five or more, or six or more) of the enzymes of the pathways described herein. Thus, a pathway within an engineered host can include all exogenous enzymes, or can include both endogenous and exogenous enzymes. Endogenous genes of the engineered hosts also can be disrupted to prevent the formation of undesirable metabolites or prevent the loss of intermediates in the pathway through other enzymes acting on such intermediates. Engineered hosts can be referred to as recombinant hosts or recombinant host cells. As described herein recombinant hosts can include nucleic acids encoding one or more of a decarboxylase, reductase, amidase, monooxygenase, oxidase, dehydrogenase, or ω-transaminase as described herein.

In addition, the production of one or more C5 building blocks can be performed in vitro using the isolated enzymes described herein, using a lysate (e.g., a cell lysate) from a host microorganism as a source of the enzymes, or using a plurality of lysates from different host microorganisms as the source of the enzymes.

### Biosynthetic Methods

The present document provides methods of producing 5-aminopentanoate in a recombinant host. The methods can include enzymatically converting lysine to cadaverine in a recombinant host using a polypeptide having decarboxylase activity; and enzymatically converting cadaverine to 5-aminopentanoate, in the recombinant host using a polypeptide having oxidase activity.

In some embodiments, the polypeptide having decarboxylase activity has at least 70% sequence identity to an amino acid sequence set forth in SEQ ID NOs: 1, 16, 17, or 18. In some embodiments, the polypeptide having decarboxylase activity is classified under EC 4.1.1.-.

In some embodiments, the polypeptide having oxidase activity has at least 70% sequence identity to an amino acid sequence set forth in SEQ ID NO: 21. In some embodiments, the polypeptide having oxidase activity is classified under EC 1.4.3.21.

The present document further provides methods of producing 5-aminopentanoate in a recombinant host. The method includes enzymatically converting lysine to 5-aminopentanamide in the recombinant host using a polypeptide having monooxygenase activity; and enzymatically converting 5-aminopentanamide to 5-aminopentanoate in the recombinant host using a polypeptide having amidase activity.

In some embodiments, the polypeptide having monooxygenase activity has at least 70% sequence identity to an amino acid sequence set forth in SEQ ID NO: 20. In some embodiments, the polypeptide having monooxygenase activity is classified under EC 1.13.12.2.

In some embodiments, the polypeptide having amidase activity has at least 70% sequence identity to an amino acid sequence set forth in SEQ ID NO: 19. In some embodiments, the polypeptide having amidase activity is classified under EC 3.5.1.30.

The present document further provides methods of producing 5-aminopentanoate in a recombinant host. The method includes enzymatically converting cadaverine to 5-aminopentanal in the recombinant host using a polypeptide having ω-transaminase activity; and enzymatically converting 5-aminopentanal to 5-aminopentanoate in the recombinant host using a polypeptide having aldehyde dehydrogenase activity.

In some embodiments, the polypeptide having ω-transaminase activity has at least 70% sequence identity to an amino acid sequence set forth in SEQ ID NOs. 8 to 13. In some embodiments, the polypeptide having ω-transaminase activity is classified under EC 2.6.1.-. In some embodiments, the polypeptide having aldehyde dehydrogenase activity is classified under EC 1.2.1.3 or EC 1.2.1.4.

In some embodiments, cadaverine can be enzymatically produced from lysine in the recombinant host using a polypeptide having decarboxylase activity. In some embodiments, the polypeptide having decarboxylase activity has at least 70% sequence identity to one of the amino acid sequences set forth in SEQ ID NOs: 1 and 16 to 18. In some embodiments, the polypeptide having decarboxylase activity is classified under EC 4.1.1.-.

In some embodiments, the method further includes enzymatically converting 5-aminopentanoate to a product selected from the group consisting of glutaric acid, 5-hydroxypentanoate, and 1,5-pentanediol. In some embodiments, 5-aminopentanoate is converted to the product using one or more polypeptides having transaminase, dehydrogenase, or carboxylate reductase activity.

In some embodiments, one or more steps of the method are performed by fermentation. In some embodiments, the host is subjected to a cultivation strategy under aerobic, anaerobic, micro-aerobic, or mixed oxygen/denitrification cultivation conditions. In some embodiments, the host is cultured under conditions of phosphate, oxygen, and/or nitrogen limitation. In some embodiments, the host is retained using a ceramic membrane to maintain a high cell density during fermentation.

In some embodiments, the principal carbon source fed to the fermentation derives from biological or non-biological feedstocks. In some embodiments, the biological feedstock is, or derives from, monosaccharides, disaccharides, lignocellulose, hemicellulose, cellulose, lignin, levulinic acid, formic acid, triglycerides, glycerol, fatty acids, agricultural waste, condensed distillers' solubles, or municipal waste. In some embodiments, the non-biological feedstock is, or derives from, natural gas, syngas, CO₂/H₂, methanol, ethanol, benzoate, non-volatile residue (NVR) caustic wash waste stream from cyclohexane oxidation processes, or terephthalic acid/isophthalic acid mixture waste streams.

In some embodiments, the host comprises one or more polypeptides having attenuated polyhydroxyalkanoate synthase, acetyl-CoA thioesterase, acetyl-CoA specific β-ketothiolase, acetoacetyl-CoA reductase, phosphotransacetylase forming acetate, acetate kinase, lactate dehydrogenase, menaquinol-fumarate oxidoreductase, 2-oxoacid decarboxylase producing isobutanol, alcohol dehydrogenase forming ethanol, triose phosphate isomerase, pyruvate decarboxylase, glucose-6-phosphate isomerase, transhydrogenase dissipating a cofactor imbalance, NADH-specific glutamate dehydrogenase, NADH/NADPH-utilizing glutamate dehydrogenase, glutaryl-CoA dehydrogenase, or acyl-CoA dehydrogenase activity.

In some embodiments, the host overexpresses one or more genes encoding a polypeptide having acetyl-CoA synthetase; 6-phosphogluconate dehydrogenase; transketolase; puridine nucleotide transhydrogenase; formate dehydrogenase; glyceraldehyde-3P-dehydrogenase; malic enzyme; glucose-6-phosphate dehydrogenase; fructose 1,6 diphosphatase; L-alanine dehydrogenase; PEP carboxylase, pyruvate carboxylase; PEP carboxykinase; PEP synthase; L-glutamate dehydrogenase specific to the NADPH used to generate a co-factor imbalance; methanol dehydrogenase; formaldehyde dehydrogenase; lysine transporter; dicarboxylate transporter; S-adenosylmethionine synthetase; 3-phosphoglycerate dehydrogenase; 3-phosphoserine aminotransferase; phosphoserine phosphatase; or a multidrug transporter activity.

In some embodiments, the host is a prokaryote, e.g., Escherichia coli, Clostridium ljungdahlii, Clostridium autoethanogenum, Clostridium kluyveri, Corynebacterium glutamicum, Cupriavidus necator, Cupriavidus metallidurans, Pseudomonas fluorescens, Pseudomonas putida, Pseudomonas oleavorans, Delftia acidovorans, Bacillus subtillis, Lactobacillus delbrueckii, Lactococcus lactis, and Rhodococcus equi.

In some embodiments, the host is a eukaryote, e.g., Aspergillus niger, Saccharomyces cerevisiae, Pichia pastoris, Yarrowia lipolytica, Issathenkia orientalis, Debaryomyces hansenii, Arxula adenoinivorans, and Kluyveromyces lactis.

### Enzymes Generating the Terminal Carboxyl Groups in the Biosynthesis of a C5 Building Block

As depicted in FIG. 2, a terminal carboxyl group can be enzymatically formed using an (i) a primary amine oxidase, (ii) a 5-aminopentanamidase, or (iii) an aldehyde dehydrogenase such as a 7-oxoheptanoate dehydrogenase, a 6-oxohexanoate dehydrogenase, or a 5-oxopentanoate dehydrogenase.

In some embodiments, the second terminal carboxyl group leading to the synthesis of glutaric acid is enzymatically formed by an aldehyde dehydrogenase classified, for example, under EC 1.2.1.3 (see, Guerrillot & Vandecasteele, Eur. J. Biochem., 1977, 81, 185-192). See, FIG. 6.

In some embodiments, the second terminal carboxyl group leading to the synthesis of glutaric acid is enzymatically formed by an aldehyde dehydrogenase classified under EC 1.2.1.- such as a glutarate semialdehyde dehydrogenase classified, for example, under EC 1.2.1.20, a succinate-semialdehyde dehydrogenase classified, for example, under EC 1.2.1.16 or EC 1.2.1.79, or an aldehyde dehydrogenase classified under EC 1.2.1.3. For example, an aldehyde dehydrogenase classified under EC 1.2.1.- can be a 5-oxopentanoate dehydrogenase such as the gene product of CpnE, a 6-oxohexanoate dehydrogenase (e.g., the gene product of ChnE from Acinetobacter sp.), or a 7-oxoheptanoate dehydrogenase (e.g., the gene product of ThnG from Sphingomonas macrogolitabida) (Iwaki et al., Appl. Environ. Microbiol., 1999, 65(11), 5158-5162; López-Sánchez et al., Appl. Environ. Microbiol., 2010, 76(1), 110-118). For example, a 6-oxohexanoate dehydrogenase can be classified under EC 1.2.1.63 such as the gene product of ChnE. For example, a 7-oxoheptanoate dehydrogenase can be classified under EC 1.2.1.-. See, FIG. 6.

In some embodiments, a terminal carboxyl group can be enzymatically formed by a primary amine oxidase classified, for example, under EC 1.4.3.21 (Saysell et al., 2002, Biochem, J., 365(Pt 3), 809-816). See FIG. 5.

In some embodiments, a terminal carboxyl group can be enzymatically formed by a 5-aminopentamidase classified, for example, under EC 3.5.1.30 (Reitz and Rodwell, 1970, J. Biol. Chem., 245(12), 3091-3096). See, FIG. 4.

### Enzymes Generating the Terminal Amine Groups in the Biosynthesis of a C5 Building Block

As depicted in FIGS. 4 and 5, terminal amine groups can be enzymatically formed using a decarboxylase such as a lysine decarboxylase, glutamate decarboxylase, ornithine decarboxylase, or an arginine decarboxylase.

In some embodiments, one terminal amine group is enzymatically formed by a decarboxylase classified, for example, under EC 4.1.1.- such as EC 4.1.1.15, EC 4.1.1.17, EC 4.1.1.18, or EC 4.1.1.19. See, FIG. 2.

### Enzymes Generating the Terminal Hydroxyl Groups in the Biosynthesis of a C5 Building Block

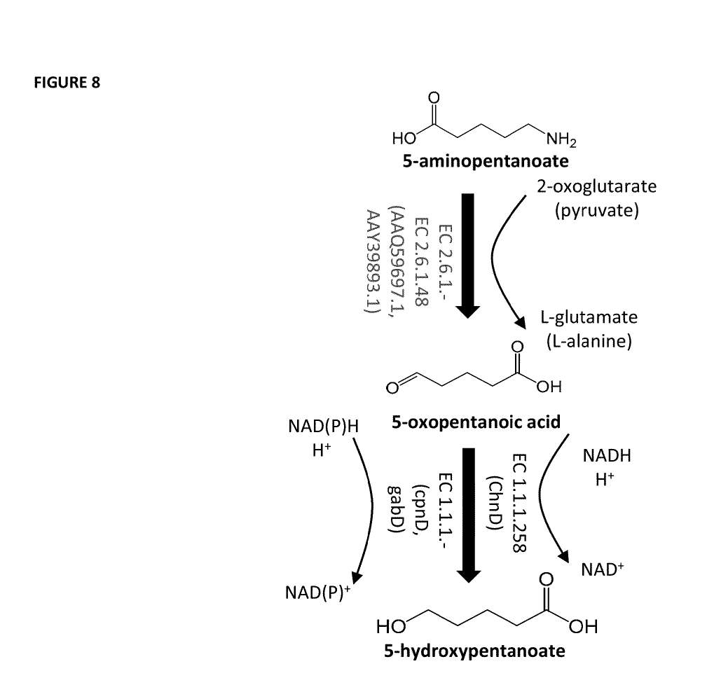

As depicted in FIGS. 8 and 9, a terminal hydroxyl group can be enzymatically formed using an alcohol dehydrogenase such as a 6-hydroxyhexanoate dehydrogenase, a 5-hydroxypentanoate dehydrogenase, or a 4-hydroxybutyrate dehydrogenase.

For example, a terminal hydroxyl group leading to the synthesis of 5-hydroxypentanoate can be enzymatically formed by a dehydrogenase classified, for example, under EC 1.1.1.- such as a 6-hydroxyhexanoate dehydrogenase classified, for example, under EC 1.1.1.258 (e.g., the gene from of ChnD), a 5-hydroxypentanoate dehydrogenase classified, for example, under EC 1.1.1.- such as the gene product of CpnD (see, for example, Iwaki et al., 2002, Appl. Environ. Microbiol., 68(11):5671-5684), a 5-hydroxypentanoate dehydrogenase from Clostridium viride, or a 4-hydroxybutyrate dehydrogenase such as gabD (see, for example, Lütke-Eversloh & Steinbiichel, 1999, FEMS Microbiology Letters, 181(1):63-71). See, FIG. 8.

A terminal hydroxyl group leading to the synthesis of 1,5 pentanediol can be enzymatically formed by an alcohol dehydrogenase classified under EC 1.1.1.- (e.g., EC 1.1.1.1, 1.1.1.2, 1.1.1.21, or 1.1.1.184). See, FIG. 9.

### Biochemical Pathways

Pathway to Cadaverine or 5-Aminopentanamide from L-Lysine

As depicted in FIG. 2, L-lysine can be converted to cadaverine by a decarboxylase classified, for example, under EC 4.1.1.- such as EC 4.1.1.15, EC 4.1.1.17, EC 4.1.1.18 or EC 4.1.1.19. For example, an Escherichia coli glutamate decarboxylase (Genbank Accession No. AAA23833.1, SEQ ID NO: 16), an Escherichia coli lysine decarboxylase (see Genbank Accession No. AAA23536.1, SEQ ID NO: 17), an Escherichia coli ornithine decarboxylase (see Genbank Accession No. AAA23536.1, SEQ ID NO: 18), or an Escherichia coli lysine decarboxylase (see Genbank Accession No. BAA21656.1, SEQ ID NO: 1) can be used to convert L-lysine to cadaverine.

As depicted in FIG. 3, L-lysine also can be converted to 5-aminopentanamide by a lysine-2-monooxygenase classified, for example, under EC 1.13.12.2 such as the gene product of davB (see Genbank Accession No. BAG54787.1, SEQ ID NO: 20). See, FIG. 3.

### Pathway to 5-Aminopentanoate Using Cadaverine or 5-Aminopentanamide as a Central Precursor

As depicted in FIG. 4, 5-aminopentanamide can be converted to 5-aminopentanoate using a 5-aminopentanamidase classified, for example, under EC 3.5.1.30 such as the gene product of davA (See Genbank Accession No. ADI95308.1, SEQ ID NO: 19).

As depicted in FIG. 5, cadaverine can be converted to 5-aminopentanoate using a primary amine oxidase classified, for example, under EC 1.4.3.21 such as the gene product of tynA (See Genbank Accession No. BAA04900.1, SEQ ID NO: 21).

As depicted in FIG. 6, cadaverine can be converted to 5-aminopentanal by a ω-transaminase classified, for example, under EC 2.6.1.- such as 2.6.1.18, EC 2.6.1.19, EC 2.6.1.29, EC 2.6.1.48, or EC 2.6.1.82 such as from a Chromobacterium violaceum (see Genbank Accession No. AAQ59697.1, SEQ ID NO: 8), a Pseudomonas aeruginosa (see Genbank Accession No. AAG08191.1, SEQ ID NO: 9), a Pseudomonas syringae (see Genbank Accession No. AAY39893.1, SEQ ID NO: 10), or an Escherichia coli (see Genbank Accession No. AAA57874.1, SEQ ID NO: 12); followed by conversion to 5-aminopentanoate using an aldehyde dehydrogenase classified under EC 1.2.1.- such as EC 1.2.1.3 or EC 1.2.1.4.

Pathway to Glutarate from 5-Aminopentanoate

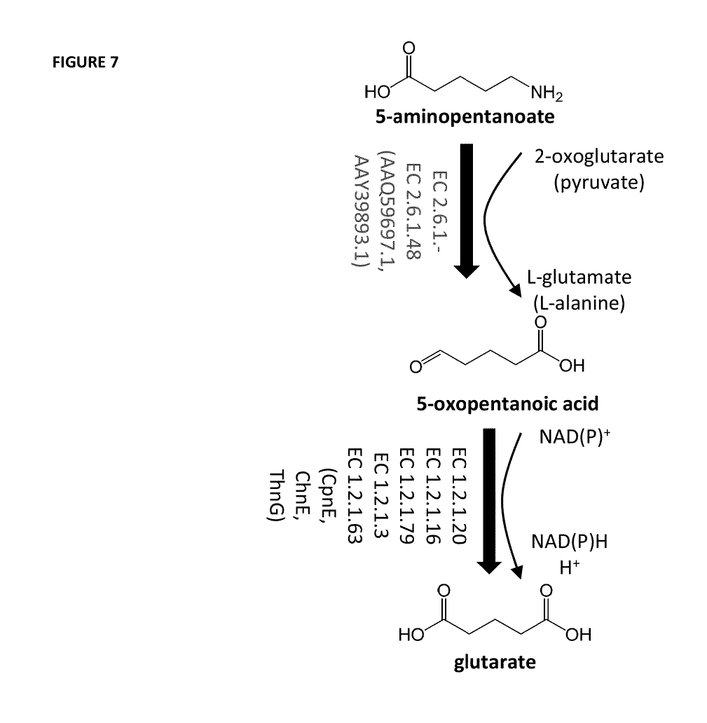

As depicted in FIG. 7, 5-aminopentanoate can be converted to 5-oxopentanoic acid using a ω-transaminase classified, for example, under EC 2.6.1.- such as EC 2.6.1.48, such as that obtained from Chromobacterium violaceum (Genbank Accession No. AAQ59697.1, SEQ ID NO: 8), Pseudomonas syringae (Genbank Accession No. AAY39893.1, SEQ ID NO: 10) or from Clostridium viride; followed by conversion to glutarate using a dehydrogenase classified under EC 1.2.1.- such as a glutarate semialdehyde dehydrogenase classified, for example, under EC 1.2.1.20, a succinate semialdehyde dehydrogenase classified, for example, under EC 1.2.1.16 or EC 1.2.1.79, an aldehyde dehydrogenase classified under EC 1.2.1.3, a 5-oxovalerate dehydrogenase such as the gene product of CpnE, a 6-oxohexanoate dehydrogenase classified under EC 1.2.1.63 (e.g., the gene product of ChnE from Acinetobacter sp.), or a 7-oxoheptanoate dehydrogenase (e.g., the gene product of ThnG from Sphingomonas macrogolitabida).

### Pathway to 5-Hydroxypentanoate Using 5-Aminopentanoate as Central Precursor

As depicted in FIG. 8, 5-aminopentanoate can be converted to 5-oxopentanoic acid using a ω-transaminase classified, for example, under EC 2.6.1.- such as EC 2.6.1.48 such as that obtained from Chromobacterium violaceum (Genbank Accession No. AAQ59697.1, SEQ ID NO: 8), Pseudomonas syringae (Genbank Accession No. AAY39893.1, SEQ ID NO: 10) or from Clostridium viride; followed by conversion to 5-hydroxypentanoate by a dehydrogenase classified, for example, under EC 1.1.1.- such as a 6-hydroxyhexanoate dehydrogenase classified, for example, under EC 1.1.1.258 (e.g., the gene from of ChnD), a 5-hydroxypentanoate dehydrogenase classified, for example, under EC 1.1.1.- such as the gene product of CpnD (see, for example, Iwaki et al., 2002, Appl. Environ. Microbiol., 68(11):5671-5684), a 5-hydroxypentanoate dehydrogenase from Clostridium viride, or a 4-hydroxybutyrate dehydrogenase such as gabD (see, for example, Lütke-Eversloh & Steinbüchel, 1999, FEMS Microbiology Letters, 181(1):63-71).

### Pathways Using 5-Hydroxypentanoate as Central Precursor to 1,5-Pentanediol

As depicted in FIG. 9, 1,5 pentanediol can be synthesized from the central precursor 5-hydroxypentanoate by conversion of 5-hydroxypentanoate to 5-hydroxypentanal by a carboxylate reductase classified, for example, under EC 1.2.99.6 such as from Mycobacterium marinum (see Genbank Accession No. ACC40567.1, SEQ ID NO: 2), Mycobacterium smegmatis (see Genbank Accession No. ABK71854.1, SEQ ID NO: 3), Segniliparus rugosus (see Genbank Accession No. EFV11917.1, SEQ ID NO: 4), Mycobacterium massiliense (see Genbank Accession No. EIV11143.1, SEQ ID NO: 6), or Segniliparus rotundus (see Genbank Accession No. ADG98140.1, SEQ ID NO: 7), in combination with a phosphopantetheine transferase enhancer (e.g., encoded by a sfp (Genbank Accession No. CAA44858.1, SEQ ID NO: 21) gene from Bacillus subtilis or npt (Genbank Accession No. ABI83656.1, SEQ ID NO: 22) gene from Nocardia), or the gene product of GriC & GriD (Suzuki et al., J. Antibiot., 2007, 60(6), 380-387); followed by conversion of 5-hydroxypentanal to 1,5 pentanediol by an alcohol dehydrogenase classified, for example, under EC 1.1.1.- such as EC 1.1.1.1, EC 1.1.1.2, EC 1.1.1.21, or EC 1.1.1.184) such as the gene product of YMR318C (Genbank Accession No. CAA90836.1) or YqhD (from E. coli, GenBank Accession No. AAA69178.1) (see, e.g., Liu et al., Microbiology, 2009, 155, 2078-2085; Larroy et al., 2002, Biochem J., 361(Pt 1), 163-172; or Jarboe, 2011, Appl. Microbiol. Biotechnol., 89(2), 249-257) or the protein having GenBank Accession No. CAA81612.1 (from Geobacillus stearothermophilus). See, FIG. 9.

### Cultivation Strategy

In some embodiments, the cultivation strategy entails achieving an aerobic, anaerobic, micro-aerobic, or mixed oxygen/denitrification cultivation condition. Enzymes characterized in vitro as being oxygen sensitive require a micro-aerobic cultivation strategy maintaining a very low dissolved oxygen concentration (See, for example, Chayabatra & Lu-Kwang, Appl. Environ. Microbiol., 2000, 66(2), 493 0 498; Wilson and Bouwer, 1997, Journal of Industrial Microbiology and Biotechnology, 18(2-3), 116-130).

In some embodiments, a cyclical cultivation strategy entails alternating between achieving an anaerobic cultivation condition and achieving an aerobic cultivation condition.

In some embodiments, the cultivation strategy entails nutrient limitation such as nitrogen, phosphate or oxygen limitation.

In some embodiments, a final electron acceptor other than oxygen such as nitrates can be utilized. In some embodiments, a cell retention strategy using, for example, ceramic membranes can be employed to achieve and maintain a high cell density during either fed-batch or continuous fermentation.

In some embodiments, the principal carbon source fed to the fermentation in the synthesis of one or more C5 building blocks can derive from biological or non-biological feedstocks.

In some embodiments, the biological feedstock can be or can derive from, monosaccharides, disaccharides, lignocellulose, hemicellulose, cellulose, lignin, levulinic acid and formic acid, triglycerides, glycerol, fatty acids, agricultural waste, condensed distillers' solubles, or municipal waste.

The efficient catabolism of crude glycerol stemming from the production of biodiesel has been demonstrated in several microorganisms such as Escherichia coli, Cupriavidus necator, Pseudomonas oleavorans, Pseudomonas putida and Yarrowia lipolytica (Lee et al., Appl. Biochem. Biotechnol., 2012, 166:1801-1813; Yang et al., Biotechnology for Biofuels, 2012, 5:13; Meijnen et al., Appl. Microbiol. Biotechnol., 2011, 90:885-893).

The efficient catabolism of lignocellulosic-derived levulinic acid has been demonstrated in several organisms such as Cupriavidus necator and Pseudomonas putida in the synthesis of 3-hydroxyvalerate via the precursor propanoyl-CoA (Jaremko and Yu, 2011, supra; Martin and Prather, J. Biotechnol., 2009, 139:61-67).

The efficient catabolism of lignin-derived aromatic compounds such as benzoate analogues has been demonstrated in several microorganisms such as Pseudomonas putida, Cupriavidus necator (Bugg et al., Current Opinion in Biotechnology, 2011, 22, 394-400; Pérez-Pantoja et al., FEMS Microbiol. Rev., 2008, 32, 736-794).

The efficient utilization of agricultural waste, such as olive mill waste water has been demonstrated in several microorganisms, including Yarrowia lipolytica (Papanikolaou et al., Bioresour. Technol., 2008, 99(7):2419-2428).

The efficient utilization of fermentable sugars such as monosaccharides and disaccharides derived from cellulosic, hemicellulosic, cane and beet molasses, cassava, corn and other agricultural sources has been demonstrated for several microorganism such as Escherichia coli, Corynebacterium glutamicum and Lactobacillus delbrueckii and Lactococcus lactis (see, e.g., Hermann et al, J. Biotechnol., 2003, 104:155-172; Wee et al., Food Technol. Biotechnol., 2006, 44(2):163-172; Ohashi et al., J. Bioscience and Bioengineering, 1999, 87(5):647-654).

The efficient utilization of furfural, derived from a variety of agricultural lignocellulosic sources, has been demonstrated for Cupriavidus necator (Li et al., Biodegradation, 2011, 22:1215-1225).

In some embodiments, the non-biological feedstock can be or can derive from natural gas, syngas, CO₂/H₂, methanol, ethanol, benzoate, non-volatile residue (NVR) or a caustic wash waste stream from cyclohexane oxidation processes, or terephthalic acid/isophthalic acid mixture waste streams.

The efficient catabolism of methanol has been demonstrated for the methylotrophic yeast Pichia pastoris.

The efficient catabolism of ethanol has been demonstrated for Clostridium kluyveri (Seedorf et al., Proc. Natl. Acad. Sci. USA, 2008, 105(6) 2128-2133).

The efficient catabolism of CO₂ and H₂, which may be derived from natural gas and other chemical and petrochemical sources, has been demonstrated for Cupriavidus necator (Prybylski et al., Energy, Sustainability and Society, 2012, 2:11).

The efficient catabolism of syngas has been demonstrated for numerous microorganisms, such as Clostridium ljungdahlii and Clostridium autoethanogenum (Köpke et al., Applied and Environmental Microbiology, 2011, 77(15):5467-5475).

The efficient catabolism of the non-volatile residue waste stream from cyclohexane processes has been demonstrated for numerous microorganisms, such as Delftia acidovorans and Cupriavidus necator (Ramsay et al., Applied and Environmental Microbiology, 1986, 52(1):152-156).

In some embodiments, the host microorganism is a prokaryote. For example, the prokaryote can be a bacterium from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia such as Delftia acidovorans; from the genus Bacillus such as Bacillus subtillis; from the genus Lactobacillus such as Lactobacillus delbrueckii; or from the genus Lactococcus such as Lactococcus lactis. Such prokaryotes also can be a source of genes to construct recombinant host cells described herein that are capable of producing one or more C5 building blocks.

In some embodiments, the host microorganism is a eukaryote. For example, the eukaryote can be a filamentous fungus, e.g., one from the genus Aspergillus such as Aspergillus niger. Alternatively, the eukaryote can be a yeast, e.g., one from the genus Saccharomyces such as Saccharomyces cerevisiae; from the genus Pichia such as Pichia pastoris; or from the genus Yarrowia such as Yarrowia lipolytica; from the genus Issatchenkia such as Issathenkia orientalis; from the genus Debaryomyces such as Debaryomyces hansenii; from the genus Arxula such as Arxula adenoinivorans; or from the genus Kluyveromyces such as Kluyveromyces lactis. Such eukaryotes also can be a source of genes to construct recombinant host cells described herein that are capable of producing one or more C5 building blocks.

### Metabolic Engineering

The present document provides methods involving less than all the steps described for all the above pathways. Such methods can involve, for example, one, two, three, four, five, six, seven, eight, nine, ten, eleven, twelve or more of such steps. Where less than all the steps are included in such a method, the first, and in some embodiments the only, step can be any one of the steps listed.

Furthermore, recombinant hosts described herein can include any combination of the above enzymes such that one or more of the steps, e.g., one, two, three, four, five, six, seven, eight, nine, ten, or more of such steps, can be performed within a recombinant host. This document provides host cells of any of the genera and species listed and genetically engineered to express one or more (e.g., two, three, four, five, six, seven, eight, nine, 10, 11, 12 or more) recombinant forms of any of the enzymes recited in the document. Thus, for example, the host cells can contain exogenous nucleic acids encoding enzymes catalyzing one or more of the steps of any of the pathways described herein.

In addition, this document recognizes that where enzymes have been described as accepting CoA-activated substrates, analogous enzyme activities associated with [acp]-bound substrates exist that are not necessarily in the same enzyme class.

Also, this document recognizes that where enzymes have been described accepting (R)-enantiomers of substrate, analogous enzyme activities associated with (S)-enantiomer substrates exist that are not necessarily in the same enzyme class.

This document also recognizes that where an enzyme is shown to accept a particular co-factor, such as NADPH, or co-substrate, such as acetyl-CoA, many enzymes are promiscuous in terms of accepting a number of different co-factors or co-substrates in catalyzing a particular enzyme activity. Also, this document recognizes that where enzymes have high specificity for e.g., a particular co-factor such as NADH, an enzyme with similar or identical activity that has high specificity for the co-factor NADPH may be in a different enzyme class.

In some embodiments, the enzymes in the pathways outlined herein are the result of enzyme engineering via non-direct or rational enzyme design approaches with aims of improving activity, improving specificity, reducing feedback inhibition, reducing repression, improving enzyme solubility, changing stereo-specificity, or changing co-factor specificity.

In some embodiments, the enzymes in the pathways outlined here can be gene dosed, i.e., overexpressed, into the resulting genetically modified organism via episomal or chromosomal integration approaches.

In some embodiments, genome-scale system biology techniques such as Flux Balance Analysis can be utilized to devise genome scale attenuation or knockout strategies for directing carbon flux to a C5 building block.

Attenuation strategies include, but are not limited to; the use of transposons, homologous recombination (double cross-over approach), mutagenesis, enzyme inhibitors and RNAi interference.

In some embodiments, fluxomic, metabolomic and transcriptomal data can be utilized to inform or support genome-scale system biology techniques, thereby devising genome scale attenuation or knockout strategies in directing carbon flux to a C5 building block.

In some embodiments, the host microorganism's tolerance to high concentrations of a C5 building block can be improved through continuous cultivation in a selective environment.

In some embodiments, the host microorganism's endogenous biochemical network can be attenuated or augmented to (1) ensure the intracellular availability of L-glutamate, (2) create a NADPH imbalance that may be balanced via the formation of one or more C5 building blocks, (3) prevent degradation of central metabolites, central precursors leading to and including one or more C5 building blocks and/or (4) ensure efficient efflux from the cell.

In some embodiments requiring the intracellular availability of L-glutamate for C5 building block synthesis, the enzymes catalyzing anaplerotic reactions supplementing the citric acid cycle intermediates are amplified.

In some embodiments requiring the intracellular availability of 2-oxoglutarate or oxaloacetate, a PEP carboxykinase or PEP carboxylase can be overexpressed in the host to generate anaplerotic carbon flux into the Krebs cycle towards 2-oxo-glutarate (Schwartz et al., 2009, Proteomics, 9, 5132-5142).

In some embodiments requiring the intracellular availability of 2-oxoglutarate or oxaloacetate, a pyruvate carboxylase can be overexpressed in the host to generated anaplerotic carbon flux into the Krebs cycle towards 2-oxoglutarate (Schwartz et al., 2009, Proteomics, 9, 5132-5142).

In some embodiments requiring the intracellular availability of 2-oxoglutarate or oxaloacetate, a PEP synthase can be overexpressed in the host to enhance the flux from pyruvate to PEP, thus increasing the carbon flux into the Krebs cycle via PEP carboxykinase or PEP carboxylase (Schwartz et al., 2009, Proteomics, 9, 5132-5142).

In some embodiments requiring the intracellular availability of 2-oxoglutarate or oxaloacetate for C5 building block synthesis, anaplerotic reactions enzymes such as phosphoenolpyruvate carboxylase (e.g., the gene product of pck), phosphoenolpyruvate carboxykinase (e.g., the gene product of ppc), the malic enzyme (e.g., the gene product of sfcA) and/or pyruvate carboxylase are overexpressed in the host organisms (Song and Lee, 2006, Enzyme Micr. Technol., 39, 352-361).

In some embodiments, carbon flux can be directed into the pentose phosphate cycle to increase the supply of NADPH by attenuating an endogenous glucose-6-phosphate isomerase (EC 5.3.1.9).

In some embodiments, carbon flux can be redirected into the pentose phosphate cycle to increase the supply of NADPH by overexpression a 6-phosphogluconate dehydrogenase and/or a transketolase (Lee et al., 2003, Biotechnology Progress, 19(5), 1444-1449).

In some embodiments, where pathways require excess NADPH co-factor in the synthesis of a C5 building block, a gene such as UdhA encoding a puridine nucleotide transhydrogenase can be overexpressed in the host organisms (Brigham et al., Advanced Biofuels and Bioproducts, 2012, Chapter 39, 1065-1090).

In some embodiments, where pathways require excess NADPH co-factor in the synthesis of a C5 Building Block, a recombinant glyceraldehyde-3-phosphate-dehydrogenase gene such as GapN can be overexpressed in the host organisms (Brigham et al., 2012, supra).

In some embodiments, where pathways require excess NADPH co-factor in the synthesis of a C5 building block, a recombinant malic enzyme gene such as macA or maeB can be overexpressed in the host organisms (Brigham et al., 2012, supra).

In some embodiments, where pathways require excess NADPH co-factor in the synthesis of a C5 building block, a recombinant glucose-6-phosphate dehydrogenase gene such as zwf can be overexpressed in the host organisms (Lim et al., J. Bioscience and Bioengineering, 2002, 93(6), 543-549).

In some embodiments, where pathways require excess NADPH co-factor in the synthesis of a C5 building block, a recombinant fructose 1,6 diphosphatase gene such as fbp can be overexpressed in the host organisms (Becker et al., J. Biotechnol., 2007, 132:99-109).

In some embodiments, where pathways require excess NADPH co-factor in the synthesis of a C5 building block, endogenous triose phosphate isomerase (EC 5.3.1.1) can be attenuated.

In some embodiments, where pathways require excess NADPH co-factor in the synthesis of a C5 building block, a recombinant glucose dehydrogenase such as the gene product of gdh can be overexpressed in the host organism (Satoh et al., J. Bioscience and Bioengineering, 2003, 95(4):335-341).

In some embodiments, endogenous enzymes facilitating the conversion of NADPH to NADH can be attenuated, such as the NADH generation cycle that may be generated via inter-conversion of glutamate dehydrogenases classified under EC 1.4.1.2 (NADH-specific) and EC 1.4.1.4 (NADPH-specific).

In some embodiments, an endogenous glutamate dehydrogenase (EC 1.4.1.3) that utilizes both NADH and NADPH as co-factors can be attenuated.

In some embodiments using hosts that naturally accumulate polyhydroxyalkanoates, the endogenous polyhydroxyalkanoate synthase enzymes can be attenuated in the host strain.

In some embodiments, a L-alanine dehydrogenase can be overexpressed in the host to regenerate L-alanine from pyruvate as an amino donor for ω-transaminase reactions.

In some embodiments, a L-glutamate dehydrogenase, a L-glutamine synthetase, or a glutamate synthase can be overexpressed in the host to regenerate L-glutamate from 2-oxoglutarate as an amino donor for ω-transaminase reactions.

In some embodiments, enzymes such as; an acyl-CoA dehydrogenase classified, for example, under EC 1.3.8.7 or EC 1.3.8.1; and/or a glutaryl-CoA dehydrogenase classified, for example, under EC 1.3.8.6 or EC 1.3.99.7 that degrade central metabolites and central precursors leading to and including C5 building blocks can be attenuated.

In some embodiments, endogenous enzymes activating C5 building blocks via Coenzyme A esterification such as CoA-ligases (e.g., a glutaryl-CoA synthetase) classified under, for example, EC 6.2.1.6 can be attenuated.

In some embodiments, the efflux of a C5 building block across the cell membrane to the extracellular media can be enhanced or amplified by genetically engineering structural modifications to the cell membrane or increasing any associated transporter activity for a C5 building block.

The efflux of cadaverine can be enhanced or amplified by overexpressing broad substrate range multidrug transporters such as Blt from Bacillus subtilis (Woolridge et al., 1997, J. Biol. Chem., 272(14):8864-8866); AcrB and AcrD from Escherichia coli (Elkins & Nikaido, 2002, J. Bacteriol., 184(23), 6490-6499), NorA from Staphylococcus aereus (Ng et al., 1994, Antimicrob Agents Chemother, 38(6), 1345-1355), or Bmr from Bacillus subtilis (Neyfakh, 1992, Antimicrob Agents Chemother, 36(2), 484-485).

The efflux of 5-aminopentanoate and cadaverine can be enhanced or amplified by overexpressing the solute transporters such as the lysE transporter from Corynebacterium glutamicum (Bellmann et al., 2001, Microbiology, 147, 1765-1774).

The efflux of glutaric acid can be enhanced or amplified by overexpressing a dicarboxylate transporter such as the SucE transporter from Corynebacterium glutamicum (Huhn et al., Appl. Microbiol. & Biotech., 89(2), 327-335).

### Producing C5 Building Blocks Using a Recombinant Host

Typically, one or more C5 building blocks can be produced by providing a host microorganism and culturing the provided microorganism with a culture medium containing a suitable carbon source as described above. In general, the culture media and/or culture conditions can be such that the microorganisms grow to an adequate density and produce a C5 building block efficiently. For large-scale production processes, any method can be used such as those described elsewhere (Manual of Industrial Microbiology and Biotechnology, 2^{nd} Edition, Editors: A. L. Demain and J. E. Davies, ASM Press; and Principles of Fermentation Technology, P. F. Stanbury and A. Whitaker, Pergamon). Briefly, a large tank (e.g., a 100 gallon, 200 gallon, 500 gallon, or more tank) containing an appropriate culture medium is inoculated with a particular microorganism. After inoculation, the microorganism is incubated to allow biomass to be produced. Once a desired biomass is reached, the broth containing the microorganisms can be transferred to a second tank. This second tank can be any size. For example, the second tank can be larger, smaller, or the same size as the first tank. Typically, the second tank is larger than the first such that additional culture medium can be added to the broth from the first tank. In addition, the culture medium within this second tank can be the same as, or different from, that used in the first tank.

Once transferred, the microorganisms can be incubated to allow for the production of a C5 building block. Once produced, any method can be used to isolate C5 building blocks. For example, C5 building blocks can be recovered selectively from the fermentation broth via adsorption processes. In the case of glutaric acid and 5-aminopentanoic acid, the resulting eluate can be further concentrated via evaporation, crystallized via evaporative and/or cooling crystallization, and the crystals recovered via centrifugation. In the case of cadaverine and 1,5-pentanediol, distillation may be employed to achieve the desired product purity.

Accordingly, the methods provided herein can be performed in a recombinant host. In some embodiments, the methods provided herein can be performed in a recombinant host by fermentation. In some embodiments, said recombinant host is subjected to a cultivation strategy under aerobic, anaerobic or, micro-aerobic cultivation conditions. In some embodiments, said recombinant host is cultured under conditions of nutrient limitation such as phosphate, nitrogen and oxygen limitation. In some embodiments, said recombinant host is retained using a ceramic membrane to maintain a high cell density during fermentation.

In some embodiments, the principal carbon source fed to the fermentation derives from biological or non-biological feedstocks. In some embodiments, the biological feedstock is, or derives from, monosaccharides, disaccharides, lignocellulose, hemicellulose, cellulose, lignin, levulinic acid, formic acid, triglycerides, glycerol, fatty acids, agricultural waste, condensed distillers' solubles, or municipal waste. In some embodiments, the non-biological feedstock is, or derives from, natural gas, syngas, CO₂/H₂, methanol, ethanol, benzoate, non-volatile residue (NVR) caustic wash waste stream from cyclohexane oxidation processes, or terephthalic acid/isophthalic acid mixture waste streams.

In some embodiments, the recombinant host is a prokaryote. In some embodiments, said prokaryote is from the genus Escherichia such as Escherichia coli; from the genus Clostridia such as Clostridium ljungdahlii, Clostridium autoethanogenum or Clostridium kluyveri; from the genus Corynebacteria such as Corynebacterium glutamicum; from the genus Cupriavidus such as Cupriavidus necator or Cupriavidus metallidurans; from the genus Pseudomonas such as Pseudomonas fluorescens, Pseudomonas putida or Pseudomonas oleavorans; from the genus Delftia acidovorans, from the genus Bacillus such as Bacillus subtillis; from the genes Lactobacillus such as Lactobacillus delbrueckii; from the genus Lactococcus such as Lactococcus lactis; or from the genus Rhodococcus such as Rhodococcus equi.

In some embodiments, the recombinant host is a eukaryote. In some embodiments, said eukaryote is from the genus Aspergillus such as Aspergillus niger; from the genus Saccharomyces such as Saccharomyces cerevisiae; from the genus Pichia such as Pichia pastoris; from the genus Yarrowia such as Yarrowia lipolytica, from the genus Issatchenkia such as Issathenkia orientalis, from the genus Debaryomyces such as Debaryomyces hansenii, from the genus Arxula such as Arxula adenoinivorans, or from the genus Kluyveromyces such as Kluyveromyces lactis.

In some embodiments, said recombinant host comprises one or more of the following attenuated enzymes: a polypeptide having polyhydroxyalkanoate synthase, acetyl-CoA thioesterase, acetyl-CoA specific β-ketothiolase, acetoacetyl-CoA reductase, phosphotransacetylase forming acetate, acetate kinase, lactate dehydrogenase, menaquinol-fumarate oxidoreductase, 2-oxoacid decarboxylase producing isobutanol, alcohol dehydrogenase forming ethanol, triose phosphate isomerase, pyruvate decarboxylase, glucose-6-phosphate isomerase, transhydrogenase dissipating a cofactor imbalance, NADH-specific glutamate dehydrogenase, NADH/NADPH-utilizing glutamate dehydrogenase, glutaryl-CoA dehydrogenase, or acyl-CoA dehydrogenase activity.

In some embodiments, said recombinant host overexpresses one or more genes encoding: a polypeptide having acetyl-CoA synthetase; 6-phosphogluconate dehydrogenase; transketolase; puridine nucleotide transhydrogenase; formate dehydrogenase; glyceraldehyde-3P-dehydrogenase; malic enzyme; glucose-6-phosphate dehydrogenase; fructose 1,6 diphosphatase; L-alanine dehydrogenase; PEP carboxylase, pyruvate carboxylase; PEP carboxykinase; PEP synthase; L-glutamate dehydrogenase specific to the NADPH used to generate a co-factor imbalance; methanol dehydrogenase, formaldehyde dehydrogenase, lysine transporter; dicarboxylate transporter; S-adenosylmethionine synthetase; 3-phosphoglycerate dehydrogenase; 3-phosphoserine aminotransferase; phosphoserine phosphatase; or a multidrug transporter activity.

In some aspects, said recombinant host comprises exogenous nucleic acids encoding a polypeptide having decarboxylase activity and a polypeptide having oxidase activity, said host producing 5-aminopentanoate. In one embodiment, said polypeptide having decarboxylase activity has at least 70% sequence identity to an amino acid sequence set forth in SEQ ID NOs: 1 and 16 to 18. In some embodiments, said polypeptide having decarboxylase activity is classified under EC 4.1.1.-. In one embodiment, said polypeptide having oxidase activity has at least 70% sequence identity to an amino acid sequence set forth in SEQ ID NO: 21. In some embodiments, said polypeptide having oxidase activity is classified under EC 1.4.3.21.

In some aspects, said recombinant host comprises exogenous nucleic acids encoding a polypeptide having monooxygenase activity and a polypeptide having amidase activity, said host producing 5-aminopentanoate. In one embodiment, said polypeptide having monooxygenase activity has at least 70% sequence identity to an amino acid sequence set forth in SEQ ID NO: 20. In some embodiments, said polypeptide having monooxygenase activity is classified under EC 1.13.12.2. In one embodiment, said polypeptide having amidase activity has at least 70% sequence identity to an amino acid sequence set forth in SEQ ID NO: 19. In some embodiments, said polypeptide having amidase activity is classified under EC 3.5.1.30.

In some aspects, said recombinant host comprises exogenous nucleic acids encoding a polypeptide having ω-transaminase activity and a polypeptide having aldehyde dehydrogenase activity, said host producing 5-aminopentanoate. In one embodiment, said polypeptide having ω-transaminase activity has at least 70% sequence identity to an amino acid sequence set forth in SEQ ID NOs. 8 to 13. In some embodiments, said polypeptide having ω-transaminase activity is classified under EC 2.6.1.-. In one embodiment, said polypeptide having aldehyde dehydrogenase activity is classified under EC 1.2.1.3 or EC 1.2.1.4.

In some embodiments, said recombinant host further comprises one or more exogenous polypeptides having ω-transaminase, alcohol dehydrogenase, aldehyde dehydrogenase, or carboxylate reductase activity.

The invention is further described in the following examples, which do not limit the scope of the invention described in the claims.

### EXAMPLES

### Example 1

### Enzyme Activity of ω-Transaminase Using Glutarate Semialdehyde as Substrate and Forming 5-Aminopentanoate

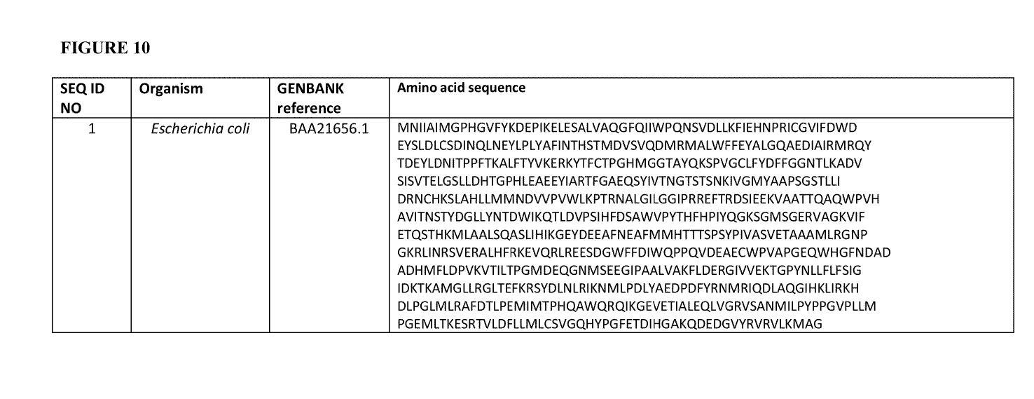

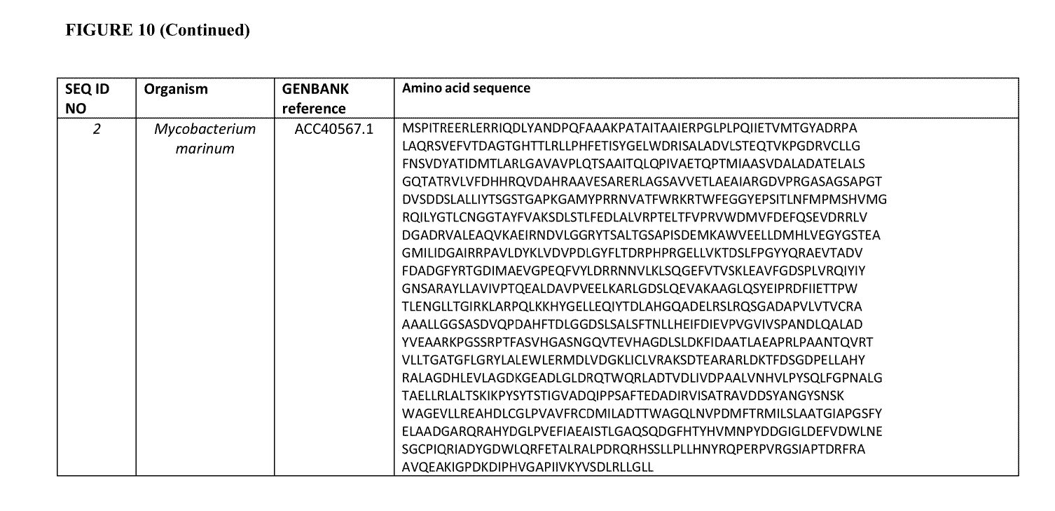

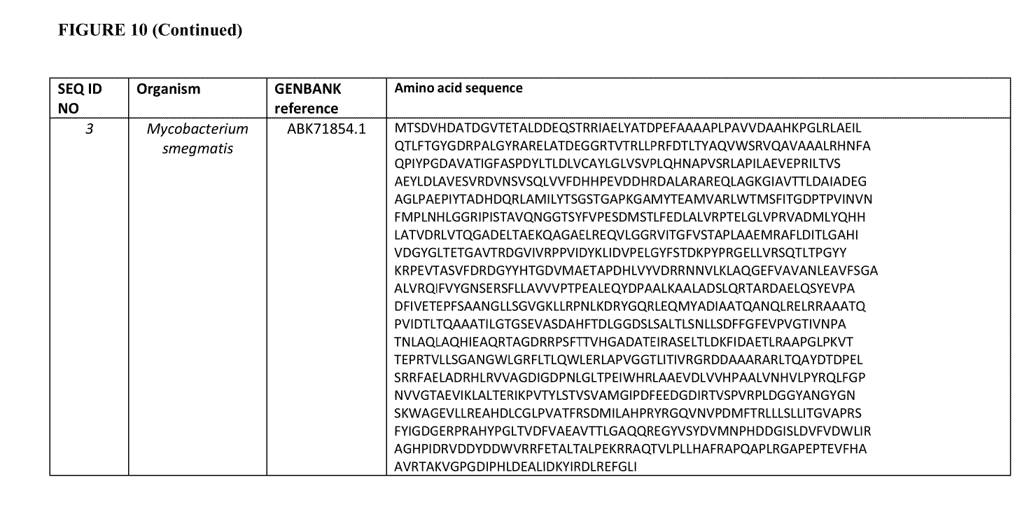

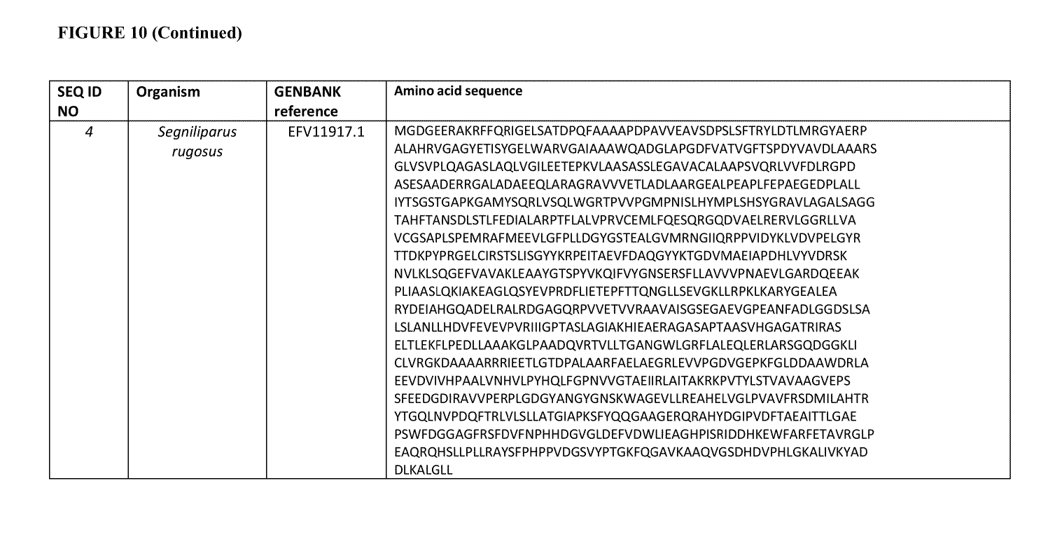

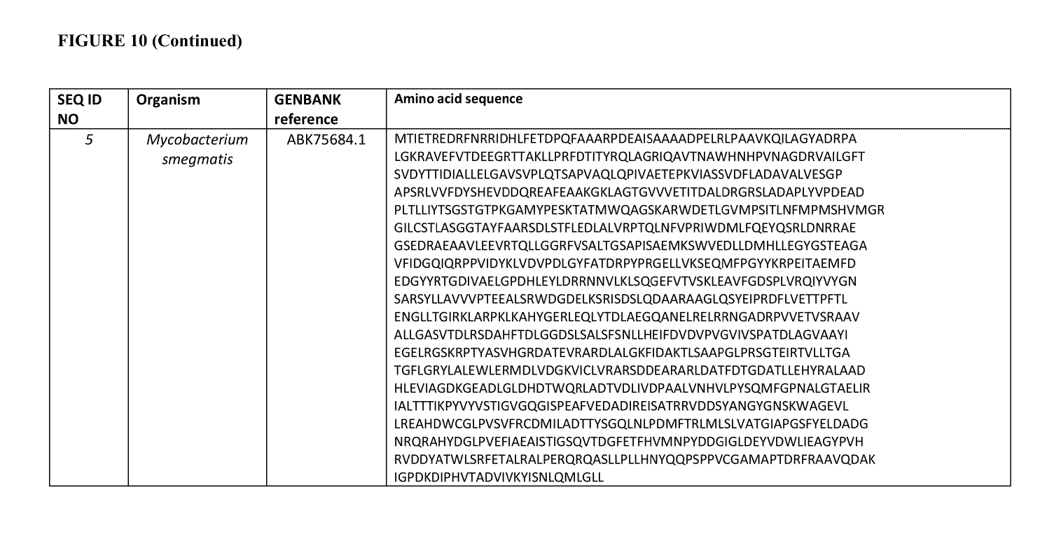

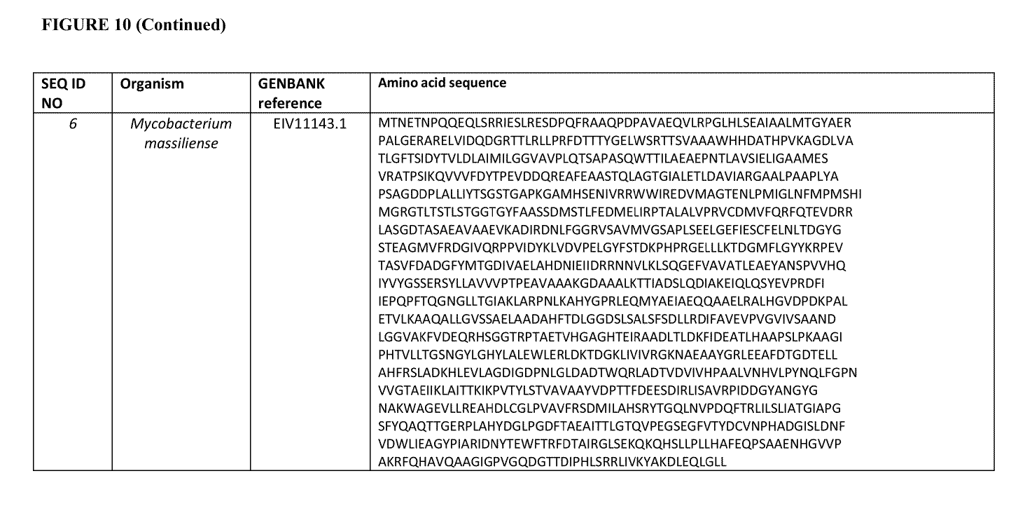

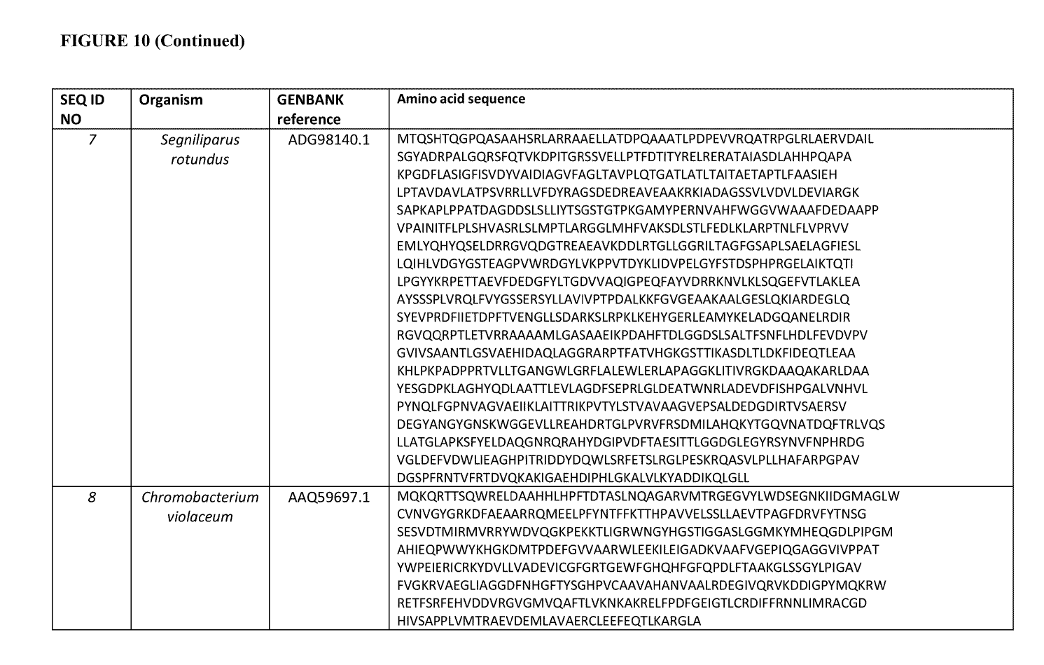

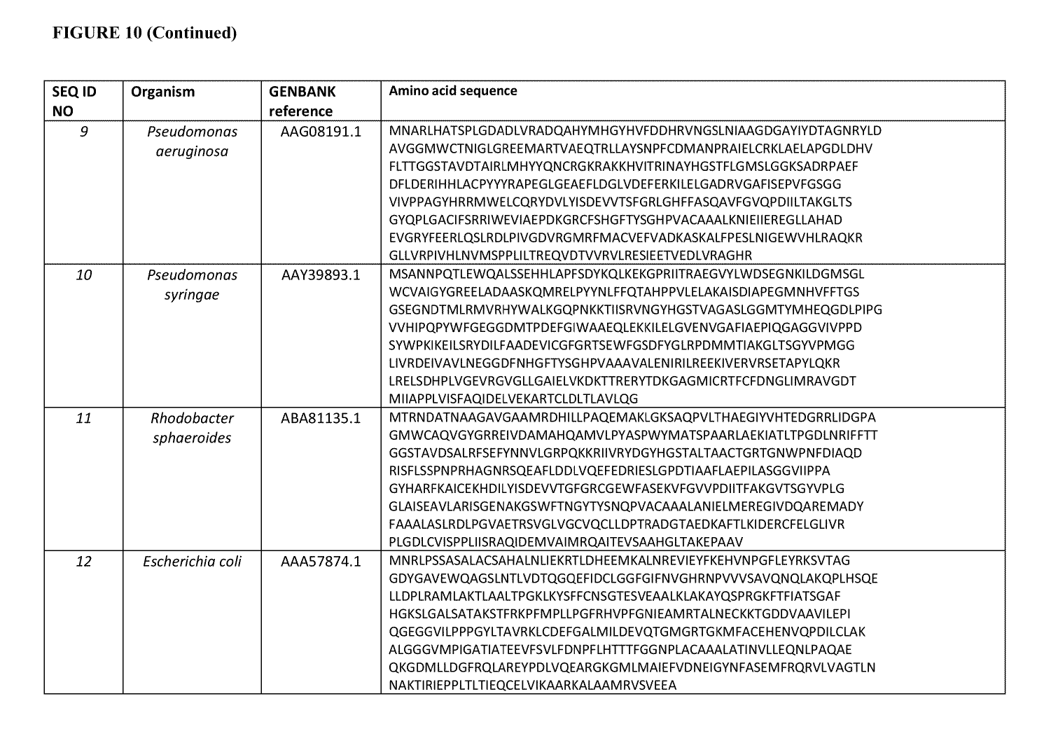

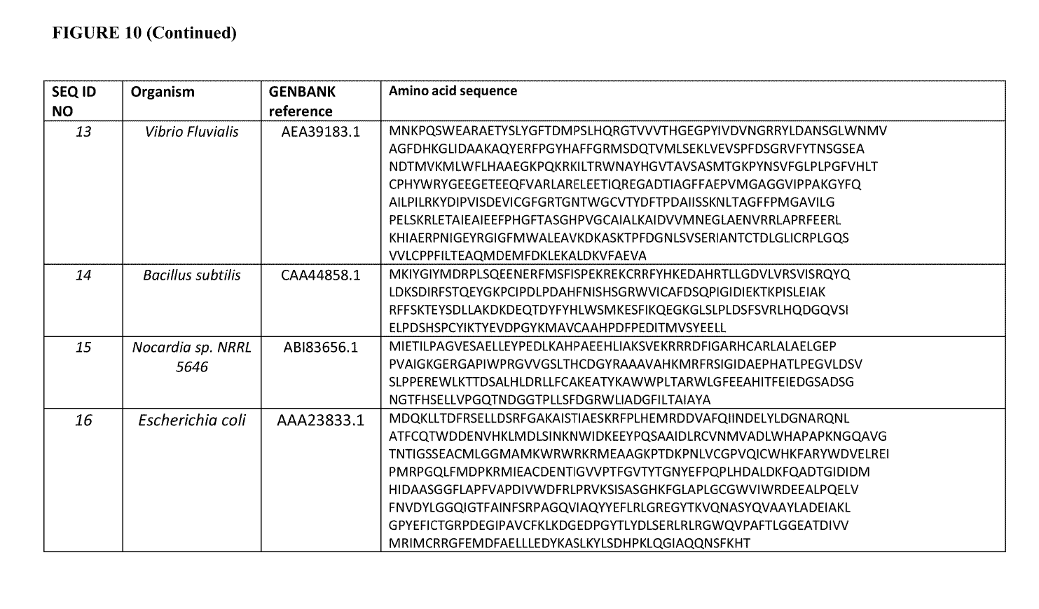

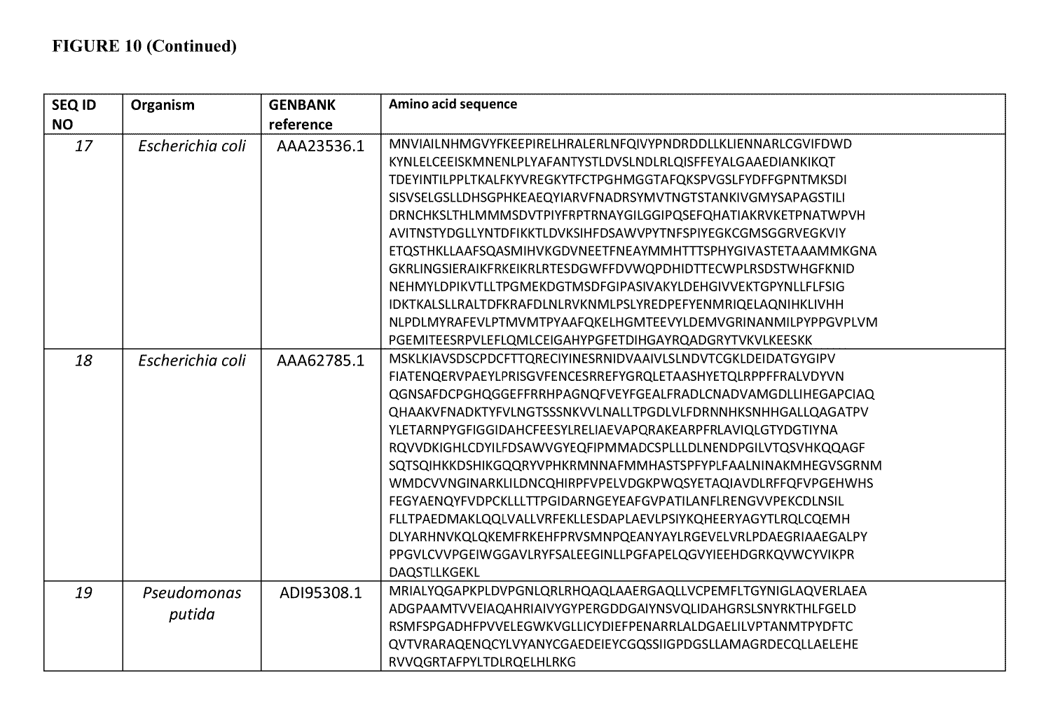

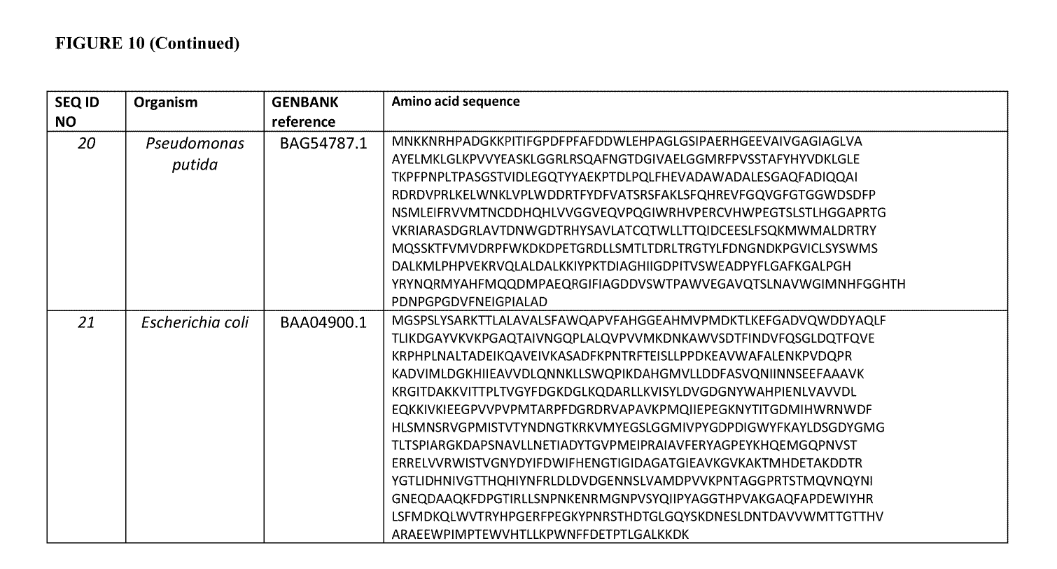

A nucleotide sequence encoding an N-terminal His-tag was added to the genes from Chromobacterium violaceum and Rhodobacter sphaeroides encoding the ω-transaminases of SEQ ID NOs: 8 and 10 respectively (see FIG. 10) such that N-terminal HIS tagged ω-transaminases could be produced. Each of the resulting modified genes was cloned into a pET21a expression vector under control of the T7 promoter and each expression vector was transformed into a BL21[DE3] E. coli host. The resulting recombinant E. coli strains were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., 5-aminopentanoate to glutarate semialdehyde) were performed in a buffer composed of a final concentration of 50 mM HEPES buffer (pH=7.5), 10 mM 5-aminopentanoate, 10 mM pyruvate and 100 μM pyridoxyl 5′ phosphate. Each enzyme activity assay reaction was initiated by adding cell free extract of the ω-transaminase gene product or the empty vector control to the assay buffer containing the 5-aminopentanoate and incubated at 25° C. for 4 hours, with shaking at 250 rpm. The formation of L-alanine from pyruvate was quantified via RP-HPLC.

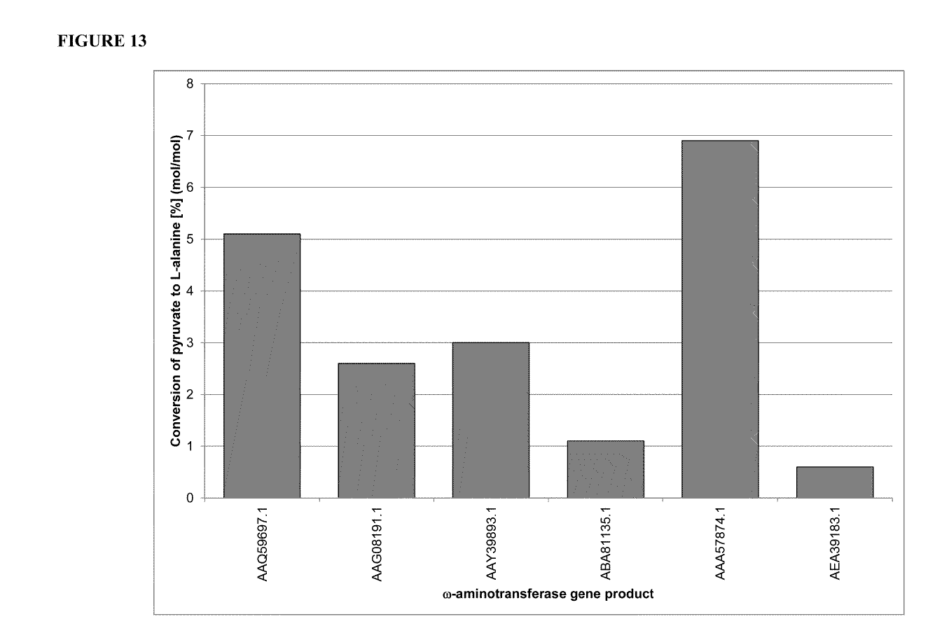

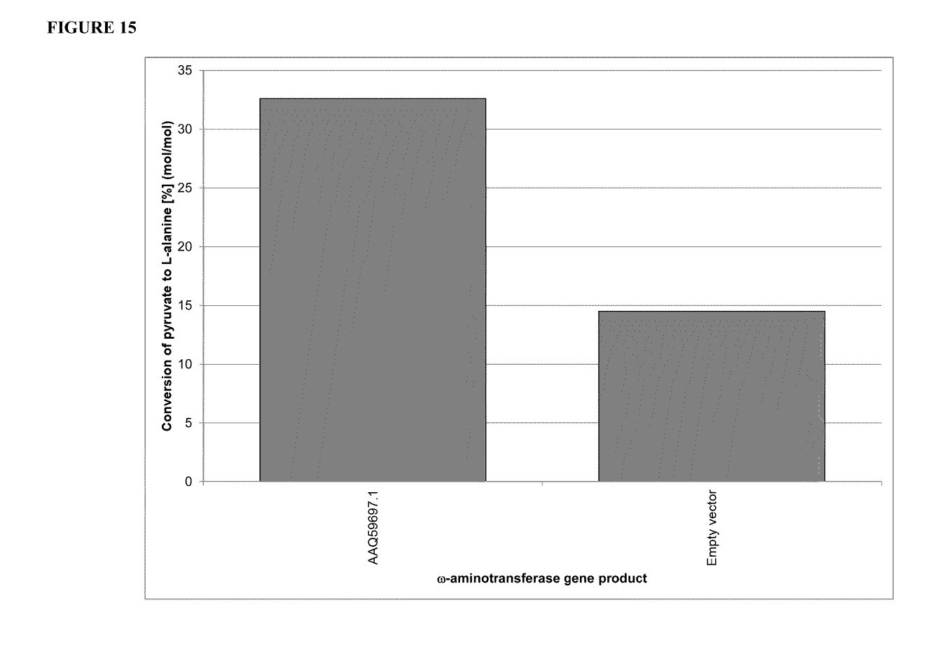

Each enzyme only control without 5-aminopentanoate demonstrated low base line conversion of pyruvate to L-alanine See, FIG. 13. The gene product of SEQ ID NO: 8, accepted 5-aminopentanoate as substrate as confirmed against the empty vector control. See, FIG. 15.

Enzyme activity in the forward direction (i.e., glutarate semialdehyde to 5-aminopentanoate) was confirmed for the transaminase of SEQ ID NO: 10. Enzyme activity assays were performed in a buffer composed of a final concentration of 50 mM HEPES buffer (pH=7.5), 10 mM glutarate semialdehyde, 10 mM L-alanine and 100 μM pyridoxyl 5′ phosphate. Each enzyme activity assay reaction was initiated by adding a cell free extract of the ω-transaminase gene product or the empty vector control to the assay buffer containing the glutarate semialdehyde and incubated at 25° C. for 4 hours, with shaking at 250 rpm. The formation of pyruvate was quantified via RP-HPLC.

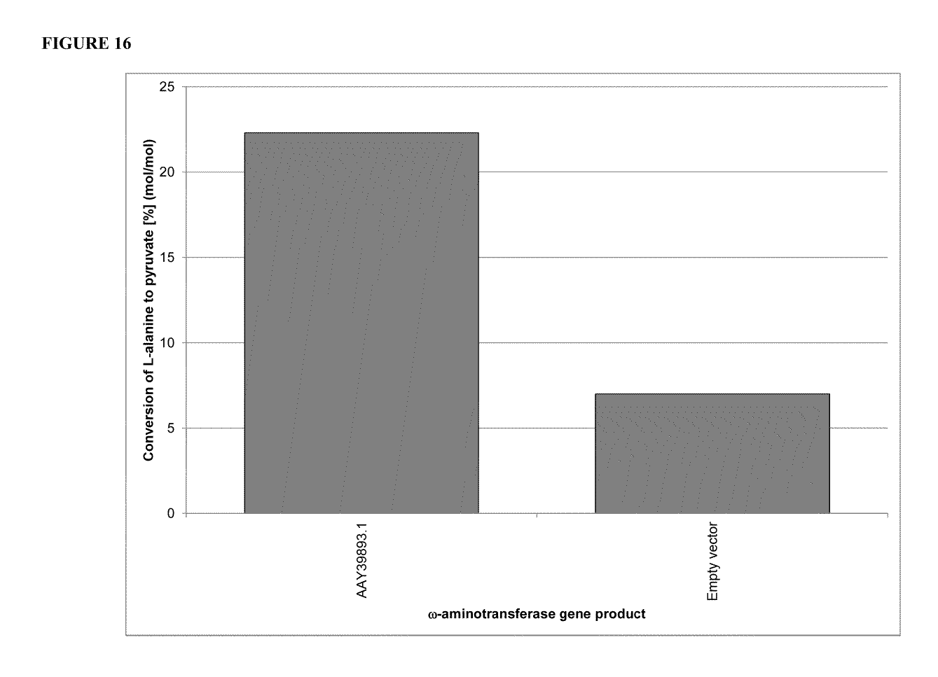

The gene product of SEQ ID NO: 10 accepted glutarate semialdehyde as substrate as confirmed against the empty vector control. See, FIG. 16. The reversibility of the ω-transaminase activity was confirmed, demonstrating that the ω-transaminases of SEQ ID NO: 8, and SEQ ID NO: 10 accepted glutarate semialdehyde as substrate and synthesized 5-aminopentanoate as a reaction product.

### Example 2

### Enzyme Activity of Carboxylate Reductase Using 5-Hydroxypentanoate as Substrate and Forming 5-Hydroxypentanal

A nucleotide sequence encoding a His-tag was added to the genes from Mycobacterium marinum, Mycobacterium smegmatis, Segniliparus rugosus, Mycobacterium massiliense, and Segniliparus rotundus that encode the carboxylate reductases of SEQ ID NOs: 2-4, 6 and 7, respectively (GenBank Accession Nos. ACC40567.1, ABK71854.1, EFV11917.1, EIV11143.1, and ADG98140.1, respectively) (see FIG. 10) such that N-terminal HIS tagged carboxylate reductases could be produced. Each of the modified genes was cloned into a pET Duet expression vector alongside a sfp gene encoding a His-tagged phosphopantetheine transferase from Bacillus subtilis, both under control of the T7 promoter. Each expression vector was transformed into a BL21[DE3] E. coli host along with the expression vectors from Example 3. Each resulting recombinant E. coli strain was cultivated at 37° C. in a 250 mL shake flask culture containing 50 mL LB media and antibiotic selection pressure, with shaking at 230 rpm. Each culture was induced overnight at 37° C. using an auto-induction media.

The pellet from each induced shake flask culture was harvested via centrifugation. Each pellet was resuspended and lysed via sonication. The cell debris was separated from the supernatant via centrifugation. The carboxylate reductases and phosphopantetheine transferase were purified from the supernatant using Ni-affinity chromatography, diluted 10-fold into 50 mM HEPES buffer (pH=7.5) and concentrated via ultrafiltration.

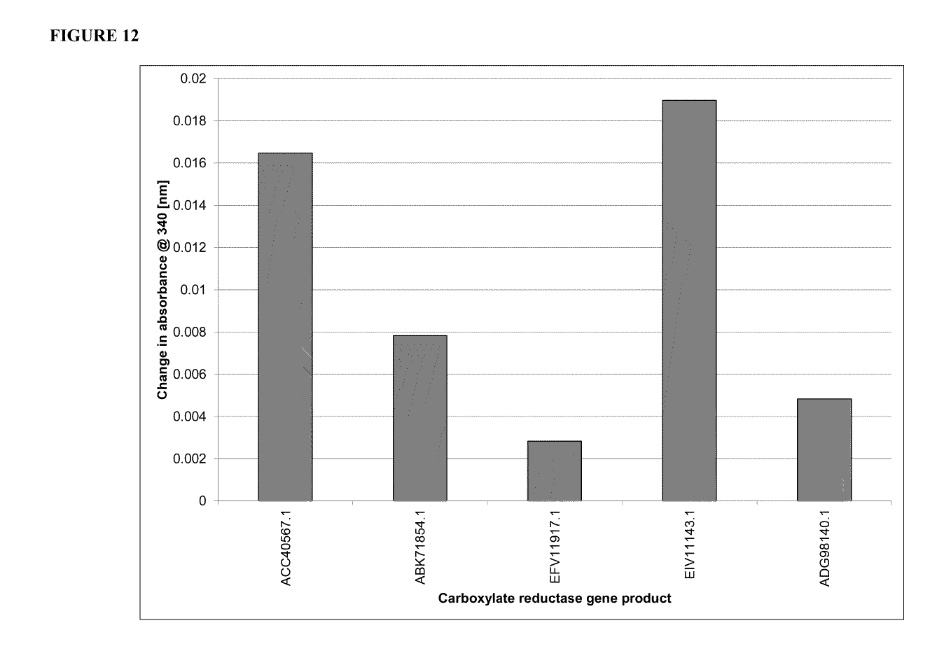

Enzyme activity (i.e., 5-hydroxypentanoate to 5-hydroxypentanal) assays were performed in triplicate in a buffer composed of a final concentration of 50 mM HEPES buffer (pH=7.5), 2 mM 5-hydroxypentanal, 10 mM MgCl₂, 1 mM ATP, and 1 mM NADPH. Each enzyme activity assay reaction was initiated by adding purified carboxylate reductase and phosphopantetheine transferase or the empty vector control to the assay buffer containing the 5-hydroxypentanoate and then incubated at room temperature for 20 minutes. The consumption of NADPH was monitored by absorbance at 340 nm. Each enzyme only control without 5-hydroxypentanoate demonstrated low base line consumption of NADPH. See, FIG. 12.

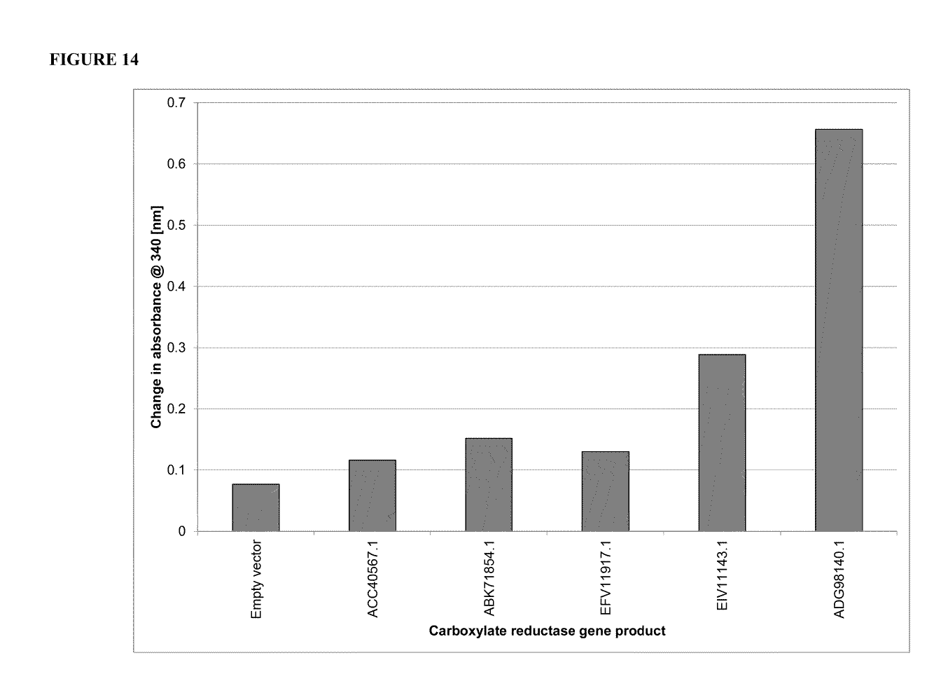

The gene products of SEQ ID NOs: 2-4, 6, and 7, enhanced by the gene product of sfp, accepted 5-hydroxypentanoate as substrate as confirmed against the empty vector control (see, FIG. 14), and synthesized 5-hydroxypentanal.

### Example 3

### Enzyme Activity of ω-Transaminase Using Cadaverine as Substrate and Forming 5-Aminopentanal

A nucleotide sequence encoding an N-terminal His-tag was added to the Chromobacterium violaceum, Pseudomonas aeruginosa, Pseudomonas syringae, and Escherichia coli genes encoding the ω-transaminases of SEQ ID NOs: 8 to 10 and 12, respectively (see, FIG. 10) such that N-terminal HIS tagged ω-transaminases could be produced. The modified genes were cloned into a pET21a expression vector under the T7 promoter. Each expression vector was transformed into a BL21[DE3] E. coli host. Each resulting recombinant E. coli strain were cultivated at 37° C. in a 250 mL shake flask culture containing 50 mL LB media and antibiotic selection pressure, with shaking at 230 rpm. Each culture was induced overnight at 16° C. using 1 mM IPTG.

The pellet from each induced shake flask culture was harvested via centrifugation. Each pellet was resuspended and lysed via sonication. The cell debris was separated from the supernatant via centrifugation and the cell free extract was used immediately in enzyme activity assays.

Enzyme activity assays in the reverse direction (i.e., cadaverine to 5-aminopentanal) were performed in a buffer composed of a final concentration of 50 mM HEPES buffer (pH=7.5), 10 mM cadaverine, 10 mM pyruvate, and 100 μM pyridoxyl 5′ phosphate. Each enzyme activity assay reaction was initiated by adding cell free extract of the ω-transaminase gene product or the empty vector control to the assay buffer containing the cadaverine and then incubated at 25° C. for 4 hours, with shaking at 250 rpm. The formation of L-alanine was quantified via RP-HPLC.

Each enzyme only control without cadaverine had low base line conversion of pyruvate to L-alanine See, FIG. 13.

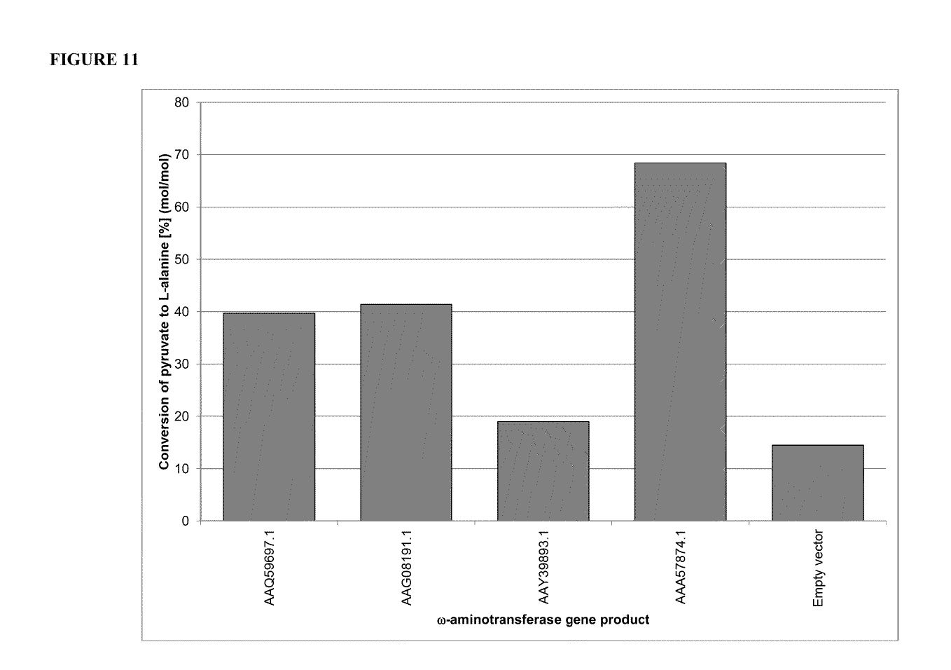

The gene products of SEQ ID NOs: 8 to 10 and 12 accepted cadaverine as substrate as confirmed against the empty vector control (see, FIG. 11) and synthesized 5-aminopentanal as reaction product. Given the reversibility of the ω-transaminase activity (see, Example 1), it can be concluded that the gene products of SEQ ID NOs: 8 to 10 and 12 accept 5-aminopentanal as substrate and form cadaverine.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of producing 5-aminopentanoate in a recombinant host, said method comprising:
enzymatically converting lysine to cadaverine in said recombinant host using a polypeptide having decarboxylase activity, said polypeptide having decarboxylase activity classified under EC 4.1.1.-. and having at least 70% sequence identity to the amino acid sequence set forth in SEQ ID NO: 1, 16, 17, or 18;
enzymatically converting cadaverine to 5-aminopentanoate in said recombinant host using a polypeptide having oxidase activity, said polypeptide having oxidase activity classified under EC 1.4.3.21 and having at least 70% sequence identity to the amino acid sequence set forth in SEQ ID NO: 21;
enzymatically converting lysine to 5-aminopentanamide in said recombinant host using a polypeptide having monooxygenase activity and enzymatically converting 5-aminopentanamide to 5-aminopentanoate in said recombinant host using a polypeptide having amidase activity, said polypeptide having monooxygenase activity classified under EC 1.13.12.2 and having at least 70% sequence identity to the amino acid sequence set forth in SEQ ID NO: 20, said polypeptide having amidase activity classified under EC 3.5.1.30 and having at least 70% sequence identity to the amino acid sequence set forth in SEQ ID NO: 19; or
enzymatically converting cadaverine to 5-aminopentanal in said recombinant host using a polypeptide having ω-transaminase activity and enzymatically converting 5-aminopentanal to 5-aminopentanoate in said recombinant host using a polypeptide having aldehyde dehydrogenase activity, said polypeptide having ω-transaminase activity classified under EC 2.6.1.- and having at least 70% sequence identity to the amino acid sequence set forth in any one of SEQ ID NOs. 8 to 13, and said polypeptide having aldehyde dehydrogenase activity is classified under EC 1.2.1.3 or EC 1.2.1.4,
the method optionally further comprising enzymatically converting 5-aminopentanoate to a product selected from the group consisting of glutaric acid, 5-hydroxypentanoate, and 1,5 pentanediol.

2. (canceled)

3. (canceled)

4. (canceled)

5. The method of claim 1, said method further comprising enzymatically converting 5-aminopentanoate to a product selected from the group consisting of glutaric acid, 5-hydroxypentanoate, and 1,5-pentanediol using one or more polypeptides selected from the group consisting of a polypeptide having transaminase activity, a polypeptide having dehydrogenase activity, and a polypeptide having carboxylate reductase activity.

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. The method of claim 1, wherein one or more steps of said method are performed by fermentation.

19. The method of claim 18, wherein said host is retained using a ceramic membrane to maintain a high cell density during fermentation.

20. The method of claim 1, wherein said host is subjected to a cultivation strategy under aerobic, anaerobic, micro-aerobic, or mixed oxygen/denitrification cultivation conditions.

21. The method of claim 1, wherein said host is cultured under conditions of phosphate, oxygen, and/or nitrogen limitation.

22. The method of claim 18, wherein the principal carbon source fed to the fermentation derives from biological or non-biological feedstocks.

23. The method of claim 22, wherein the biological feedstock is, or derives from, monosaccharides, disaccharides, lignocellulose, hemicellulose, cellulose, lignin, levulinic acid, formic acid, triglycerides, glycerol, fatty acids, agricultural waste, condensed distillers' solubles, or municipal waste and the non-biological feedstock is, or derives from, natural gas, syngas, CO2/H2, methanol, ethanol, benzoate, non-volatile residue (NVR) caustic wash waste stream from cyclohexane oxidation processes, or terephthalic acid/isophthalic acid mixture waste streams.

24. (canceled)

25. The method of claim 18, wherein said host comprises one or more polypeptides having attenuated polyhydroxyalkanoate synthase, acetyl-CoA thioesterase, acetyl-CoA specific β-ketothiolase, acetoacetyl-CoA reductase, phosphotransacetylase forming acetate, acetate kinase, lactate dehydrogenase, menaquinol-fumarate oxidoreductase, 2-oxoacid decarboxylase producing isobutanol, alcohol dehydrogenase forming ethanol, triose phosphate isomerase, pyruvate decarboxylase, glucose-6-phosphate isomerase, transhydrogenase dissipating a cofactor imbalance, NADH-specific glutamate dehydrogenase, NADH/NADPH-utilizing glutamate dehydrogenase, glutaryl-CoA dehydrogenase, or acyl-CoA dehydrogenase activity.

26. The method of claim 18, wherein said host overexpresses one or more genes encoding a polypeptide having acetyl-CoA synthetase; 6-phosphogluconate dehydrogenase; transketolase; puridine nucleotide transhydrogenase; formate dehydrogenase; glyceraldehyde-3P-dehydrogenase; malic enzyme; glucose-6-phosphate dehydrogenase; fructose 1,6 diphosphatase; L-alanine dehydrogenase; PEP carboxylase, pyruvate carboxylase; PEP carboxykinase; PEP synthase; L-glutamate dehydrogenase specific to the NADPH used to generate a co-factor imbalance; methanol dehydrogenase, formaldehyde dehydrogenase, lysine transporter; dicarboxylate transporter; S-adenosylmethionine synthetase; 3-phosphoglycerate dehydrogenase; 3-phosphoserine aminotransferase; phosphoserine phosphatase; or a multidrug transporter activity.

27. The method of claim 1, wherein the host is a prokaryote selected from the group consisting of Escherichia; Clostridia; Corynebacteria; Cupriavidus; Pseudomonas; Delftia; Bacilluss; Lactobacillus; Lactococcus; and Rhodococcus, or a eukaryote selected from the group consisting of Aspergillus, Saccharomyces, Pichia, Yarrowia, Issatchenkia, Debaryomyces, Arxula, and Kluyveromyces.

28. (canceled)

29. (canceled)

30. (canceled)

31. (canceled)

32. (canceled)

33. A recombinant host comprising:
at least one exogenous nucleic acid encoding a polypeptide having decarboxylase activity and a polypeptide having oxidase activity, said host producing 5-aminopentanoate, said polypeptide having decarboxylase activity classified under EC 4.1.1.- and having at least 70% sequence identity to the amino acid sequence set forth in SEQ ID NO: 1, 16, 17, or 18, said polypeptide having oxidase activity classified under EC 1.4.3.21 and having at least 70% sequence identity to the amino acid sequence set forth in SEQ ID NO: 21;
at least one exogenous nucleic acid encoding a polypeptide having monooxygenase activity and a polypeptide having amidase activity, said host producing 5-aminopentanoate, said polypeptide having amidase activity classified under EC 3.5.1.30 and having at least 70% sequence identity to the amino acid sequence set forth in SEQ ID NO: 19, said polypeptide having monooxygenase activity classified under EC 1.13.12.2 and having at least 70% sequence identity to the amino acid sequence set forth in SEQ ID NO: 20; or
at least one exogenous nucleic acid encoding a polypeptide having ω-transaminase activity and a polypeptide having aldehyde dehydrogenase activity, said host producing 5-aminopentanoate, said polypeptide having ω-transaminase activity classified under EC 2.6.1.- and having at least 70% sequence identity to the amino acid sequence set forth in any one of SEQ ID NOs. 8 to 13, and said polypeptide having aldehyde dehydrogenase activity classified under EC 1.2.1.3 or EC 1.2.1.4.

34. (canceled)

35. (canceled)

36. The recombinant host of claim 33, said host further comprising one or more exogenous polypeptides selected from the group consisting of a polypeptide having ω-transaminase activity, a polypeptide having alcohol dehydrogenase activity, a polypeptide having aldehyde dehydrogenase activity, and a polypeptide having carboxylate reductase activity, said host further producing one or more of glutaric acid, 5-hydroxypentanoate, and 1,5-pentanediol.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. The recombinant host of claim 33, wherein the host is a prokaryote selected from the group consisting of Escherichia; Clostridia; Corynebacteria; Cupriavidus; Pseudomonas; Delftia; Bacilluss; Lactobacillus; Lactococcus; and Rhodococcus, or a eukaryote selected from the group consisting of Aspergillus, Saccharomyces, Pichia, Yarrowia, Issatchenkia, Debaryomyces, Arxula, and Kluyveromyces.

49. (canceled)

50. (canceled)

51. (canceled)

52. (canceled)

53. (canceled)

54. A bio-derived product, bio-based product or fermentation-derived product, wherein said product comprises:
i. a composition comprising at least one bio-derived, bio-based or fermentation-derived compound according to claim 1,
ii. a bio-derived, bio-based or fermentation-derived polymer comprising the bio-derived, bio-based or fermentation-derived composition or compound of i., or a combination thereof,
iii. a bio-derived, bio-based or fermentation-derived resin comprising the bio-derived, bio-based or fermentation-derived compound or bio-derived, bio-based or fermentation-derived composition of i. or a combination thereof or the bio-derived, bio-based or fermentation-derived polymer of ii. or a combination thereof,
iv. a molded substance obtained by molding the bio-derived, bio-based or fermentation-derived polymer of ii. or the bio-derived, bio-based or fermentation-derived resin of iii., or any a combination thereof,
v. a bio-derived, bio-based or fermentation-derived formulation comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., or bio-derived, bio-based or fermentation-derived molded substance of iv, or a combination thereof, or
vi. a bio-derived, bio-based or fermentation-derived semi-solid or a non-semi-solid stream, comprising the bio-derived, bio-based or fermentation-derived composition of i., bio-derived, bio-based or fermentation-derived compound of i., bio-derived, bio-based or fermentation-derived polymer of ii., bio-derived, bio-based or fermentation-derived resin of iii., bio-derived, bio-based or fermentation-derived formulation of v., or bio-derived, bio-based or fermentation-derived molded substance of iv., or a combination thereof.


SEQUENCE LISTING (21 sequences)

SEQ ID NO: 1 - protein, 713 aa, Escherichia coli
MNIIAIMGPHGVFYKDEPIKELESALVAQGFQIIWPQNSVDLLKFIEHNPRICGVIFDWDEYSLDLCSDINQLNEYLPLYAFINTHSTMDVSVQDMRMALWFFEYALGQAEDIAIRMRQYTDEYLDNITPPFTKALFTYVKERKYTFCTPGHMGGTAYQKSPVGCLFYDFFGGNTLKADVSISVTELGSLLDHTGPHLEAEEYIARTFGAEQSYIVTNGTSTSNKIVGMYAAPSGSTLLIDRNCHKSLAHLLMMNDVVPVWLKPTRNALGILGGIPRREFTRDSIEEKVAATTQAQWPVHAVITNSTYDGLLYNTDWIKQTLDVPSIHFDSAWVPYTHFHPIYQGKSGMSGERVAGKVIFETQSTHKMLAALSQASLIHIKGEYDEEAFNEAFMMHTTTSPSYPIVASVETAAAMLRGNPGKRLINRSVERALHFRKEVQRLREESDGWFFDIWQPPQVDEAECWPVAPGEQWHGFNDADADHMFLDPVKVTILTPGMDEQGNMSEEGIPAALVAKFLDERGIVVEKTGPYNLLFLFSIGIDKTKAMGLLRGLTEFKRSYDLNLRIKNMLPDLYAEDPDFYRNMRIQDLAQGIHKLIRKHDLPGLMLRAFDTLPEMIMTPHQAWQRQIKGEVETIALEQLVGRVSANMILPYPPGVPLLMPGEMLTKESRTVLDFLLMLCSVGQHYPGFETDIHGAKQDEDGVYRVRVLKMAG

SEQ ID NO: 2 - protein, 1174 aa, Mycobacterium marinum
MSPITREERLERRIQDLYANDPQFAAAKPATAITAAIERPGLPLPQIIETVMTGYADRPALAQRSVEFVTDAGTGHTTLRLLPHFETISYGELWDRISALADVLSTEQTVKPGDRVCLLGFNSVDYATIDMTLARLGAVAVPLQTSAAITQLQPIVAETQPTMIAASVDALADATELALSGQTATRVLVFDHHRQVDAHRAAVESARERLAGSAVVETLAEAIARGDVPRGASAGSAPGTDVSDDSLALLIYTSGSTGAPKGAMYPRRNVATFWRKRTWFEGGYEPSITLNFMPMSHVMGRQILYGTLCNGGTAYFVAKSDLSTLFEDLALVRPTELTFVPRVWDMVFDEFQSEVDRRLVDGADRVALEAQVKAEIRNDVLGGRYTSALTGSAPISDEMKAWVEELLDMHLVEGYGSTEAGMILIDGAIRRPAVLDYKLVDVPDLGYFLTDRPHPRGELLVKTDSLFPGYYQRAEVTADVFDADGFYRTGDIMAEVGPEQFVYLDRRNNVLKLSQGEFVTVSKLEAVFGDSPLVRQIYIYGNSARAYLLAVIVPTQEALDAVPVEELKARLGDSLQEVAKAAGLQSYEIPRDFIIETTPWTLENGLLTGIRKLARPQLKKHYGELLEQIYTDLAHGQADELRSLRQSGADAPVLVTVCRAAAALLGGSASDVQPDAHFTDLGGDSLSALSFTNLLHEIFDIEVPVGVIVSPANDLQALADYVEAARKPGSSRPTFASVHGASNGQVTEVHAGDLSLDKFIDAATLAEAPRLPAANTQVRTVLLTGATGFLGRYLALEWLERMDLVDGKLICLVRAKSDTEARARLDKTFDSGDPELLAHYRALAGDHLEVLAGDKGEADLGLDRQTWQRLADTVDLIVDPAALVNHVLPYSQLFGPNALGTAELLRLALTSKIKPYSYTSTIGVADQIPPSAFTEDADIRVISATRAVDDSYANGYSNSKWAGEVLLREAHDLCGLPVAVFRCDMILADTTWAGQLNVPDMFTRMILSLAATGIAPGSFYELAADGARQRAHYDGLPVEFIAEAISTLGAQSQDGFHTYHVMNPYDDGIGLDEFVDWLNESGCPIQRIADYGDWLQRFETALRALPDRQRHSSLLPLLHNYRQPERPVRGSIAPTDRFRAAVQEAKIGPDKDIPHVGAPIIVKYVSDLRLLGLL

SEQ ID NO: 3 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 4 - protein, 1148 aa, Segniliparus rugosus
MGDGEERAKRFFQRIGELSATDPQFAAAAPDPAVVEAVSDPSLSFTRYLDTLMRGYAERPALAHRVGAGYETISYGELWARVGAIAAAWQADGLAPGDFVATVGFTSPDYVAVDLAAARSGLVSVPLQAGASLAQLVGILEETEPKVLAASASSLEGAVACALAAPSVQRLVVFDLRGPDASESAADERRGALADAEEQLARAGRAVVVETLADLAARGEALPEAPLFEPAEGEDPLALLIYTSGSTGAPKGAMYSQRLVSQLWGRTPVVPGMPNISLHYMPLSHSYGRAVLAGALSAGGTAHFTANSDLSTLFEDIALARPTFLALVPRVCEMLFQESQRGQDVAELRERVLGGRLLVAVCGSAPLSPEMRAFMEEVLGFPLLDGYGSTEALGVMRNGIIQRPPVIDYKLVDVPELGYRTTDKPYPRGELCIRSTSLISGYYKRPEITAEVFDAQGYYKTGDVMAEIAPDHLVYVDRSKNVLKLSQGEFVAVAKLEAAYGTSPYVKQIFVYGNSERSFLLAVVVPNAEVLGARDQEEAKPLIAASLQKIAKEAGLQSYEVPRDFLIETEPFTTQNGLLSEVGKLLRPKLKARYGEALEARYDEIAHGQADELRALRDGAGQRPVVETVVRAAVAISGSEGAEVGPEANFADLGGDSLSALSLANLLHDVFEVEVPVRIIIGPTASLAGIAKHIEAERAGASAPTAASVHGAGATRIRASELTLEKFLPEDLLAAAKGLPAADQVRTVLLTGANGWLGRFLALEQLERLARSGQDGGKLICLVRGKDAAAARRRIEETLGTDPALAARFAELAEGRLEVVPGDVGEPKFGLDDAAWDRLAEEVDVIVHPAALVNHVLPYHQLFGPNVVGTAEIIRLAITAKRKPVTYLSTVAVAAGVEPSSFEEDGDIRAVVPERPLGDGYANGYGNSKWAGEVLLREAHELVGLPVAVFRSDMILAHTRYTGQLNVPDQFTRLVLSLLATGIAPKSFYQQGAAGERQRAHYDGIPVDFTAEAITTLGAEPSWFDGGAGFRSFDVFNPHHDGVGLDEFVDWLIEAGHPISRIDDHKEWFARFETAVRGLPEAQRQHSLLPLLRAYSFPHPPVDGSVYPTGKFQGAVKAAQVGSDHDVPHLGKALIVKYADDLKALGLL

SEQ ID NO: 5 - protein, 1168 aa, Mycobacterium smegmatis
MTIETREDRFNRRIDHLFETDPQFAAARPDEAISAAAADPELRLPAAVKQILAGYADRPALGKRAVEFVTDEEGRTTAKLLPRFDTITYRQLAGRIQAVTNAWHNHPVNAGDRVAILGFTSVDYTTIDIALLELGAVSVPLQTSAPVAQLQPIVAETEPKVIASSVDFLADAVALVESGPAPSRLVVFDYSHEVDDQREAFEAAKGKLAGTGVVVETITDALDRGRSLADAPLYVPDEADPLTLLIYTSGSTGTPKGAMYPESKTATMWQAGSKARWDETLGVMPSITLNFMPMSHVMGRGILCSTLASGGTAYFAARSDLSTFLEDLALVRPTQLNFVPRIWDMLFQEYQSRLDNRRAEGSEDRAEAAVLEEVRTQLLGGRFVSALTGSAPISAEMKSWVEDLLDMHLLEGYGSTEAGAVFIDGQIQRPPVIDYKLVDVPDLGYFATDRPYPRGELLVKSEQMFPGYYKRPEITAEMFDEDGYYRTGDIVAELGPDHLEYLDRRNNVLKLSQGEFVTVSKLEAVFGDSPLVRQIYVYGNSARSYLLAVVVPTEEALSRWDGDELKSRISDSLQDAARAAGLQSYEIPRDFLVETTPFTLENGLLTGIRKLARPKLKAHYGERLEQLYTDLAEGQANELRELRRNGADRPVVETVSRAAVALLGASVTDLRSDAHFTDLGGDSLSALSFSNLLHEIFDVDVPVGVIVSPATDLAGVAAYIEGELRGSKRPTYASVHGRDATEVRARDLALGKFIDAKTLSAAPGLPRSGTEIRTVLLTGATGFLGRYLALEWLERMDLVDGKVICLVRARSDDEARARLDATFDTGDATLLEHYRALAADHLEVIAGDKGEADLGLDHDTWQRLADTVDLIVDPAALVNHVLPYSQMFGPNALGTAELIRIALTTTIKPYVYVSTIGVGQGISPEAFVEDADIREISATRRVDDSYANGYGNSKWAGEVLLREAHDWCGLPVSVFRCDMILADTTYSGQLNLPDMFTRLMLSLVATGIAPGSFYELDADGNRQRAHYDGLPVEFIAEAISTIGSQVTDGFETFHVMNPYDDGIGLDEYVDWLIEAGYPVHRVDDYATWLSRFETALRALPERQRQASLLPLLHNYQQPSPPVCGAMAPTDRFRAAVQDAKIGPDKDIPHVTADVIVKYISNLQMLGLL

SEQ ID NO: 6 - protein, 1185 aa, Mycobacterium massiliense
MTNETNPQQEQLSRRIESLRESDPQFRAAQPDPAVAEQVLRPGLHLSEAIAALMTGYAERPALGERARELVIDQDGRTTLRLLPRFDTTTYGELWSRTTSVAAAWHHDATHPVKAGDLVATLGFTSIDYTVLDLAIMILGGVAVPLQTSAPASQWTTILAEAEPNTLAVSIELIGAAMESVRATPSIKQVVVFDYTPEVDDQREAFEAASTQLAGTGIALETLDAVIARGAALPAAPLYAPSAGDDPLALLIYTSGSTGAPKGAMHSENIVRRWWIREDVMAGTENLPMIGLNFMPMSHIMGRGTLTSTLSTGGTGYFAASSDMSTLFEDMELIRPTALALVPRVCDMVFQRFQTEVDRRLASGDTASAEAVAAEVKADIRDNLFGGRVSAVMVGSAPLSEELGEFIESCFELNLTDGYGSTEAGMVFRDGIVQRPPVIDYKLVDVPELGYFSTDKPHPRGELLLKTDGMFLGYYKRPEVTASVFDADGFYMTGDIVAELAHDNIEIIDRRNNVLKLSQGEFVAVATLEAEYANSPVVHQIYVYGSSERSYLLAVVVPTPEAVAAAKGDAAALKTTIADSLQDIAKEIQLQSYEVPRDFIIEPQPFTQGNGLLTGIAKLARPNLKAHYGPRLEQMYAEIAEQQAAELRALHGVDPDKPALETVLKAAQALLGVSSAELAADAHFTDLGGDSLSALSFSDLLRDIFAVEVPVGVIVSAANDLGGVAKFVDEQRHSGGTRPTAETVHGAGHTEIRAADLTLDKFIDEATLHAAPSLPKAAGIPHTVLLTGSNGYLGHYLALEWLERLDKTDGKLIVIVRGKNAEAAYGRLEEAFDTGDTELLAHFRSLADKHLEVLAGDIGDPNLGLDADTWQRLADTVDVIVHPAALVNHVLPYNQLFGPNVVGTAEIIKLAITTKIKPVTYLSTVAVAAYVDPTTFDEESDIRLISAVRPIDDGYANGYGNAKWAGEVLLREAHDLCGLPVAVFRSDMILAHSRYTGQLNVPDQFTRLILSLIATGIAPGSFYQAQTTGERPLAHYDGLPGDFTAEAITTLGTQVPEGSEGFVTYDCVNPHADGISLDNFVDWLIEAGYPIARIDNYTEWFTRFDTAIRGLSEKQKQHSLLPLLHAFEQPSAAENHGVVPAKRFQHAVQAAGIGPVGQDGTTDIPHLSRRLIVKYAKDLEQLGLL

SEQ ID NO: 7 - protein, 1186 aa, Segniliparus rotundus
MTQSHTQGPQASAAHSRLARRAAELLATDPQAAATLPDPEVVRQATRPGLRLAERVDAILSGYADRPALGQRSFQTVKDPITGRSSVELLPTFDTITYRELRERATAIASDLAHHPQAPAKPGDFLASIGFISVDYVAIDIAGVFAGLTAVPLQTGATLATLTAITAETAPTLFAASIEHLPTAVDAVLATPSVRRLLVFDYRAGSDEDREAVEAAKRKIADAGSSVLVDVLDEVIARGKSAPKAPLPPATDAGDDSLSLLIYTSGSTGTPKGAMYPERNVAHFWGGVWAAAFDEDAAPPVPAINITFLPLSHVASRLSLMPTLARGGLMHFVAKSDLSTLFEDLKLARPTNLFLVPRVVEMLYQHYQSELDRRGVQDGTREAEAVKDDLRTGLLGGRILTAGFGSAPLSAELAGFIESLLQIHLVDGYGSTEAGPVWRDGYLVKPPVTDYKLIDVPELGYFSTDSPHPRGELAIKTQTILPGYYKRPETTAEVFDEDGFYLTGDVVAQIGPEQFAYVDRRKNVLKLSQGEFVTLAKLEAAYSSSPLVRQLFVYGSSERSYLLAVIVPTPDALKKFGVGEAAKAALGESLQKIARDEGLQSYEVPRDFIIETDPFTVENGLLSDARKSLRPKLKEHYGERLEAMYKELADGQANELRDIRRGVQQRPTLETVRRAAAAMLGASAAEIKPDAHFTDLGGDSLSALTFSNFLHDLFEVDVPVGVIVSAANTLGSVAEHIDAQLAGGRARPTFATVHGKGSTTIKASDLTLDKFIDEQTLEAAKHLPKPADPPRTVLLTGANGWLGRFLALEWLERLAPAGGKLITIVRGKDAAQAKARLDAAYESGDPKLAGHYQDLAATTLEVLAGDFSEPRLGLDEATWNRLADEVDFISHPGALVNHVLPYNQLFGPNVAGVAEIIKLAITTRIKPVTYLSTVAVAAGVEPSALDEDGDIRTVSAERSVDEGYANGYGNSKWGGEVLLREAHDRTGLPVRVFRSDMILAHQKYTGQVNATDQFTRLVQSLLATGLAPKSFYELDAQGNRQRAHYDGIPVDFTAESITTLGGDGLEGYRSYNVFNPHRDGVGLDEFVDWLIEAGHPITRIDDYDQWLSRFETSLRGLPESKRQASVLPLLHAFARPGPAVDGSPFRNTVFRTDVQKAKIGAEHDIPHLGKALVLKYADDIKQLGLL

SEQ ID NO: 8 - protein, 459 aa, Chromobacterium violaceum
MQKQRTTSQWRELDAAHHLHPFTDTASLNQAGARVMTRGEGVYLWDSEGNKIIDGMAGLWCVNVGYGRKDFAEAARRQMEELPFYNTFFKTTHPAVVELSSLLAEVTPAGFDRVFYTNSGSESVDTMIRMVRRYWDVQGKPEKKTLIGRWNGYHGSTIGGASLGGMKYMHEQGDLPIPGMAHIEQPWWYKHGKDMTPDEFGVVAARWLEEKILEIGADKVAAFVGEPIQGAGGVIVPPATYWPEIERICRKYDVLLVADEVICGFGRTGEWFGHQHFGFQPDLFTAAKGLSSGYLPIGAVFVGKRVAEGLIAGGDFNHGFTYSGHPVCAAVAHANVAALRDEGIVQRVKDDIGPYMQKRWRETFSRFEHVDDVRGVGMVQAFTLVKNKAKRELFPDFGEIGTLCRDIFFRNNLIMRACGDHIVSAPPLVMTRAEVDEMLAVAERCLEEFEQTLKARGLA

SEQ ID NO: 9 - protein, 468 aa, Pseudomonas aeruginosa
MNARLHATSPLGDADLVRADQAHYMHGYHVFDDHRVNGSLNIAAGDGAYIYDTAGNRYLDAVGGMWCTNIGLGREEMARTVAEQTRLLAYSNPFCDMANPRAIELCRKLAELAPGDLDHVFLTTGGSTAVDTAIRLMHYYQNCRGKRAKKHVITRINAYHGSTFLGMSLGGKSADRPAEFDFLDERIHHLACPYYYRAPEGLGEAEFLDGLVDEFERKILELGADRVGAFISEPVFGSGGVIVPPAGYHRRMWELCQRYDVLYISDEVVTSFGRLGHFFASQAVFGVQPDIILTAKGLTSGYQPLGACIFSRRIWEVIAEPDKGRCFSHGFTYSGHPVACAAALKNIEIIEREGLLAHADEVGRYFEERLQSLRDLPIVGDVRGMRFMACVEFVADKASKALFPESLNIGEWVHLRAQKRGLLVRPIVHLNVMSPPLILTREQVDTVVRVLRESIEETVEDLVRAGHR

SEQ ID NO: 10 - protein, 454 aa, Pseudomonas syringae
MSANNPQTLEWQALSSEHHLAPFSDYKQLKEKGPRIITRAEGVYLWDSEGNKILDGMSGLWCVAIGYGREELADAASKQMRELPYYNLFFQTAHPPVLELAKAISDIAPEGMNHVFFTGSGSEGNDTMLRMVRHYWALKGQPNKKTIISRVNGYHGSTVAGASLGGMTYMHEQGDLPIPGVVHIPQPYWFGEGGDMTPDEFGIWAAEQLEKKILELGVENVGAFIAEPIQGAGGVIVPPDSYWPKIKEILSRYDILFAADEVICGFGRTSEWFGSDFYGLRPDMMTIAKGLTSGYVPMGGLIVRDEIVAVLNEGGDFNHGFTYSGHPVAAAVALENIRILREEKIVERVRSETAPYLQKRLRELSDHPLVGEVRGVGLLGAIELVKDKTTRERYTDKGAGMICRTFCFDNGLIMRAVGDTMIIAPPLVISFAQIDELVEKARTCLDLTLAVLQG

SEQ ID NO: 11 - protein, 467 aa, Rhodobacter sphaeroides
MTRNDATNAAGAVGAAMRDHILLPAQEMAKLGKSAQPVLTHAEGIYVHTEDGRRLIDGPAGMWCAQVGYGRREIVDAMAHQAMVLPYASPWYMATSPAARLAEKIATLTPGDLNRIFFTTGGSTAVDSALRFSEFYNNVLGRPQKKRIIVRYDGYHGSTALTAACTGRTGNWPNFDIAQDRISFLSSPNPRHAGNRSQEAFLDDLVQEFEDRIESLGPDTIAAFLAEPILASGGVIIPPAGYHARFKAICEKHDILYISDEVVTGFGRCGEWFASEKVFGVVPDIITFAKGVTSGYVPLGGLAISEAVLARISGENAKGSWFTNGYTYSNQPVACAAALANIELMEREGIVDQAREMADYFAAALASLRDLPGVAETRSVGLVGCVQCLLDPTRADGTAEDKAFTLKIDERCFELGLIVRPLGDLCVISPPLIISRAQIDEMVAIMRQAITEVSAAHGLTAKEPAAV

SEQ ID NO: 12 - protein, 459 aa, Escherichia coli
MNRLPSSASALACSAHALNLIEKRTLDHEEMKALNREVIEYFKEHVNPGFLEYRKSVTAGGDYGAVEWQAGSLNTLVDTQGQEFIDCLGGFGIFNVGHRNPVVVSAVQNQLAKQPLHSQELLDPLRAMLAKTLAALTPGKLKYSFFCNSGTESVEAALKLAKAYQSPRGKFTFIATSGAFHGKSLGALSATAKSTFRKPFMPLLPGFRHVPFGNIEAMRTALNECKKTGDDVAAVILEPIQGEGGVILPPPGYLTAVRKLCDEFGALMILDEVQTGMGRTGKMFACEHENVQPDILCLAKALGGGVMPIGATIATEEVFSVLFDNPFLHTTTFGGNPLACAAALATINVLLEQNLPAQAEQKGDMLLDGFRQLAREYPDLVQEARGKGMLMAIEFVDNEIGYNFASEMFRQRVLVAGTLNNAKTIRIEPPLTLTIEQCELVIKAARKALAAMRVSVEEA

SEQ ID NO: 13 - protein, 453 aa, Vibrio Fluvialis
MNKPQSWEARAETYSLYGFTDMPSLHQRGTVVVTHGEGPYIVDVNGRRYLDANSGLWNMVAGFDHKGLIDAAKAQYERFPGYHAFFGRMSDQTVMLSEKLVEVSPFDSGRVFYTNSGSEANDTMVKMLWFLHAAEGKPQKRKILTRWNAYHGVTAVSASMTGKPYNSVFGLPLPGFVHLTCPHYWRYGEEGETEEQFVARLARELEETIQREGADTIAGFFAEPVMGAGGVIPPAKGYFQAILPILRKYDIPVISDEVICGFGRTGNTWGCVTYDFTPDAIISSKNLTAGFFPMGAVILGPELSKRLETAIEAIEEFPHGFTASGHPVGCAIALKAIDVVMNEGLAENVRRLAPRFEERLKHIAERPNIGEYRGIGFMWALEAVKDKASKTPFDGNLSVSERIANTCTDLGLICRPLGQSVVLCPPFILTEAQMDEMFDKLEKALDKVFAEVA

SEQ ID NO: 14 - protein, 224 aa, Bacillus subtilis
MKIYGIYMDRPLSQEENERFMSFISPEKREKCRRFYHKEDAHRTLLGDVLVRSVISRQYQLDKSDIRFSTQEYGKPCIPDLPDAHFNISHSGRWVICAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 15 - protein, 222 aa, Nocardia sp. NRRL 5646
MIETILPAGVESAELLEYPEDLKAHPAEEHLIAKSVEKRRRDFIGARHCARLALAELGEPPVAIGKGERGAPIWPRGVVGSLTHCDGYRAAAVAHKMRFRSIGIDAEPHATLPEGVLDSVSLPPEREWLKTTDSALHLDRLLFCAKEATYKAWWPLTARWLGFEEAHITFEIEDGSADSGNGTFHSELLVPGQTNDGGTPLLSFDGRWLIADGFILTAIAYA

SEQ ID NO: 16 - protein, 466 aa, Escherichia coli
MDQKLLTDFRSELLDSRFGAKAISTIAESKRFPLHEMRDDVAFQIINDELYLDGNARQNLATFCQTWDDENVHKLMDLSINKNWIDKEEYPQSAAIDLRCVNMVADLWHAPAPKNGQAVGTNTIGSSEACMLGGMAMKWRWRKRMEAAGKPTDKPNLVCGPVQICWHKFARYWDVELREIPMRPGQLFMDPKRMIEACDENTIGVVPTFGVTYTGNYEFPQPLHDALDKFQADTGIDIDMHIDAASGGFLAPFVAPDIVWDFRLPRVKSISASGHKFGLAPLGCGWVIWRDEEALPQELVFNVDYLGGQIGTFAINFSRPAGQVIAQYYEFLRLGREGYTKVQNASYQVAAYLADEIAKLGPYEFICTGRPDEGIPAVCFKLKDGEDPGYTLYDLSERLRLRGWQVPAFTLGGEATDIVVMRIMCRRGFEMDFAELLLEDYKASLKYLSDHPKLQGIAQQNSFKHT

SEQ ID NO: 17 - protein, 715 aa, Escherichia coli
MNVIAILNHMGVYFKEEPIRELHRALERLNFQIVYPNDRDDLLKLIENNARLCGVIFDWDKYNLELCEEISKMNENLPLYAFANTYSTLDVSLNDLRLQISFFEYALGAAEDIANKIKQTTDEYINTILPPLTKALFKYVREGKYTFCTPGHMGGTAFQKSPVGSLFYDFFGPNTMKSDISISVSELGSLLDHSGPHKEAEQYIARVFNADRSYMVTNGTSTANKIVGMYSAPAGSTILIDRNCHKSLTHLMMMSDVTPIYFRPTRNAYGILGGIPQSEFQHATIAKRVKETPNATWPVHAVITNSTYDGLLYNTDFIKKTLDVKSIHFDSAWVPYTNFSPIYEGKCGMSGGRVEGKVIYETQSTHKLLAAFSQASMIHVKGDVNEETFNEAYMMHTTTSPHYGIVASTETAAAMMKGNAGKRLINGSIERAIKFRKEIKRLRTESDGWFFDVWQPDHIDTTECWPLRSDSTWHGFKNIDNEHMYLDPIKVTLLTPGMEKDGTMSDFGIPASIVAKYLDEHGIVVEKTGPYNLLFLFSIGIDKTKALSLLRALTDFKRAFDLNLRVKNMLPSLYREDPEFYENMRIQELAQNIHKLIVHHNLPDLMYRAFEVLPTMVMTPYAAFQKELHGMTEEVYLDEMVGRINANMILPYPPGVPLVMPGEMITEESRPVLEFLQMLCEIGAHYPGFETDIHGAYRQADGRYTVKVLKEESKK

SEQ ID NO: 18 - protein, 732 aa, Escherichia coli
MSKLKIAVSDSCPDCFTTQRECIYINESRNIDVAAIVLSLNDVTCGKLDEIDATGYGIPVFIATENQERVPAEYLPRISGVFENCESRREFYGRQLETAASHYETQLRPPFFRALVDYVNQGNSAFDCPGHQGGEFFRRHPAGNQFVEYFGEALFRADLCNADVAMGDLLIHEGAPCIAQQHAAKVFNADKTYFVLNGTSSSNKVVLNALLTPGDLVLFDRNNHKSNHHGALLQAGATPVYLETARNPYGFIGGIDAHCFEESYLRELIAEVAPQRAKEARPFRLAVIQLGTYDGTIYNARQVVDKIGHLCDYILFDSAWVGYEQFIPMMADCSPLLLDLNENDPGILVTQSVHKQQAGFSQTSQIHKKDSHIKGQQRYVPHKRMNNAFMMHASTSPFYPLFAALNINAKMHEGVSGRNMWMDCVVNGINARKLILDNCQHIRPFVPELVDGKPWQSYETAQIAVDLRFFQFVPGEHWHSFEGYAENQYFVDPCKLLLTTPGIDARNGEYEAFGVPATILANFLRENGVVPEKCDLNSILFLLTPAEDMAKLQQLVALLVRFEKLLESDAPLAEVLPSIYKQHEERYAGYTLRQLCQEMHDLYARHNVKQLQKEMFRKEHFPRVSMNPQEANYAYLRGEVELVRLPDAEGRIAAEGALPYPPGVLCVVPGEIWGGAVLRYFSALEEGINLLPGFAPELQGVYIEEHDGRKQVWCYVIKPRDAQSTLLKGEKL

SEQ ID NO: 19 - protein, 264 aa, Pseudomonas putida
MRIALYQGAPKPLDVPGNLQRLRHQAQLAAERGAQLLVCPEMFLTGYNIGLAQVERLAEAADGPAAMTVVEIAQAHRIAIVYGYPERGDDGAIYNSVQLIDAHGRSLSNYRKTHLFGELDRSMFSPGADHFPVVELEGWKVGLLICYDIEFPENARRLALDGAELILVPTANMTPYDFTCQVTVRARAQENQCYLVYANYCGAEDEIEYCGQSSIIGPDGSLLAMAGRDECQLLAELEHERVVQGRTAFPYLTDLRQELHLRKG

SEQ ID NO: 20 - protein, 560 aa, Pseudomonas putida
MNKKNRHPADGKKPITIFGPDFPFAFDDWLEHPAGLGSIPAERHGEEVAIVGAGIAGLVAAYELMKLGLKPVVYEASKLGGRLRSQAFNGTDGIVAELGGMRFPVSSTAFYHYVDKLGLETKPFPNPLTPASGSTVIDLEGQTYYAEKPTDLPQLFHEVADAWADALESGAQFADIQQAIRDRDVPRLKELWNKLVPLWDDRTFYDFVATSRSFAKLSFQHREVFGQVGFGTGGWDSDFPNSMLEIFRVVMTNCDDHQHLVVGGVEQVPQGIWRHVPERCVHWPEGTSLSTLHGGAPRTGVKRIARASDGRLAVTDNWGDTRHYSAVLATCQTWLLTTQIDCEESLFSQKMWMALDRTRYMQSSKTFVMVDRPFWKDKDPETGRDLLSMTLTDRLTRGTYLFDNGNDKPGVICLSYSWMSDALKMLPHPVEKRVQLALDALKKIYPKTDIAGHIIGDPITVSWEADPYFLGAFKGALPGHYRYNQRMYAHFMQQDMPAEQRGIFIAGDDVSWTPAWVEGAVQTSLNAVWGIMNHFGGHTHPDNPGPGDVFNEIGPIALAD

SEQ ID NO: 21 - protein, 757 aa, Escherichia coli
MGSPSLYSARKTTLALAVALSFAWQAPVFAHGGEAHMVPMDKTLKEFGADVQWDDYAQLFTLIKDGAYVKVKPGAQTAIVNGQPLALQVPVVMKDNKAWVSDTFINDVFQSGLDQTFQVEKRPHPLNALTADEIKQAVEIVKASADFKPNTRFTEISLLPPDKEAVWAFALENKPVDQPRKADVIMLDGKHIIEAVVDLQNNKLLSWQPIKDAHGMVLLDDFASVQNIINNSEEFAAAVKKRGITDAKKVITTPLTVGYFDGKDGLKQDARLLKVISYLDVGDGNYWAHPIENLVAVVDLEQKKIVKIEEGPVVPVPMTARPFDGRDRVAPAVKPMQIIEPEGKNYTITGDMIHWRNWDFHLSMNSRVGPMISTVTYNDNGTKRKVMYEGSLGGMIVPYGDPDIGWYFKAYLDSGDYGMGTLTSPIARGKDAPSNAVLLNETIADYTGVPMEIPRAIAVFERYAGPEYKHQEMGQPNVSTERRELVVRWISTVGNYDYIFDWIFHENGTIGIDAGATGIEAVKGVKAKTMHDETAKDDTRYGTLIDHNIVGTTHQHIYNFRLDLDVDGENNSLVAMDPVVKPNTAGGPRTSTMQVNQYNIGNEQDAAQKFDPGTIRLLSNPNKENRMGNPVSYQIIPYAGGTHPVAKGAQFAPDEWIYHRLSFMDKQLWVTRYHPGERFPEGKYPNRSTHDTGLGQYSKDNESLDNTDAVVWMTTGTTHVARAEEWPIMPTEWVHTLLKPWNFFDETPTLGALKKDK